(19) United States
(12) Patent Application Publication
(10) Pub. No.: US 2026/0069713 A1
(43) Pub. Date: Mar. 12, 2026

(54) Lipid-Coated Nanoparticles

(71) Applicant: Vivasor, Inc., San Diego, CA (US)

(72) Inventors: Hui XIE, San Diego, CA (US); Hua WANG, San Diego, CA (US); Yan HE, San Diego, CA (US); Haotian SUN, San Diego, CA (US); Wan-Lin LIM, San Diego, CA (US); Jonathan D. WANG, San Diego, CA (US); Chonling Liu, San Diego, CA (US); Dahai Jiang, San Diego, CA (US)

(73) Assignee: Vivasor, Inc., San Diego, CA (US)

(21) Appl. No.: 19/107,408

(22) PCT Filed: Aug. 28, 2023
(86) PCT No.: PCT/US23/73004

§ 371 Date: Feb. 27, 2025

Related U.S. Application Data
(60) Provisional application No. 63/402,020, filed on Aug. 29, 2022.

(51) Int. Cl. A61K 48/00; C12N 15/88

(52) U.S. Cl. CPC A61K 48/0041; A61K 48/005; A61K 48/0091; C12N 15/88

(57) ABSTRACT

Provided herein, inter alia, lipid-coated nanoparticles, lipid-coated lipid nanoparticles (LNPs), and methods of making lipid-coated lipid nanoparticles.

### CROSS REFERENCE TO RELATED APPLICATIONS

This application claims the benefit of priority of U.S. Provisional Patent Application No. 63/402,020, filed Aug. 29, 2022, which application is hereby incorporated by reference in its entirety.

Throughout this application various publications, patents, and/or patent applications are referenced. The disclosures of the publications, patents and/or patent applications are hereby incorporated by reference in their entireties into this application in order to more fully describe the state of the art to which this disclosure pertains.

### TECHNICAL FIELD

Throughout this application various publications, patents, and/or patent applications are referenced. The disclosures of the publications, patents and/or patent applications are hereby incorporated by reference in their entireties into this application in order to more fully describe the state of the art to which this disclosure pertains.

### BACKGROUND

The present disclosure provides lipid-coated nanoparticles, e.g., to facilitate intracellular payload delivery (e.g., of mRNA, DNA, siRNA, oligonucleotides, amplified RNA, plasmids, ribozymes, aptamers, etc.). The present disclosure further provides a method for making lipid-coated nanoparticles, and in particular lipid-coated lipid nanoparticles (LC-LNPs).

### INTRODUCTION AND SUMMARY

Many macromolecules, such as nucleic acid molecules, and other molecules such as imaging agents and small molecule therapeutics, cannot easily cross cell membranes because of their size, charge, and/or hydrophilicity. Delivery has therefore been one of the major challenges for such therapeutics, e.g., antisense payloads and mRNA technology. A formulation containing payload molecules (e.g., nucleic acid molecules, imaging agents, and/or small molecule therapeutics) for intracellular delivery not only must (1) protect the payload from enzymatic and non-enzymatic degradation and (2) provide appropriate biodistribution of the formulation, but also (3) allow cellular uptake or internalization of the formulation and (4) facilitate delivery of the payload to the interior of the cell. Consequently, payload delivery remains a challenge due to low efficiency or cytotoxicity.

Liposomes, the earliest generation of lipid-based nanoparticles, can entrap hydrophilic payloads in their aqueous interior, and hydrophobic payloads in their lipid bilayer. Although liposomes present an attractive delivery system due to their flexible physicochemical and biophysical properties, which can be easily manipulated, some biological challenges still remain. One such challenge is the relatively low transfection efficiency provided by liposomes.

Lipid nanoparticles (LNPs) have emerged as promising vehicles to deliver a variety of payloads across cell membranes (Tenchov R. et al., 2021, Nano 15:16982-17015). Existing LNPs are commonly composed of natural lipids and have been considered pharmacologically inactive and minimally toxic. The lipids used to prepare LNPs are able to improve drug absorption relative to liposomes by, for example, increasing solubilization capacity and enhancing membrane permeability.

Much effort has been devoted to identifying novel compositions that can enhance intracellular delivery of therapeutic agents using lipid nanoparticles, which can be adapted to a scalable, consistent, and cost-effective manufacturing process (WO 2021007278; WO 2015057751; WO 2019028387; Chen L. et al, 2017, Anal. Chem. 89:6936-6939; Han X. et al., 2021, Nat. Commun. 12:7233-7238; and Zhao S. et al., 2019, Front. Pharmacol. 10: article 102).

Despite the advantages of LNPs, as therapeutic agent delivery vehicles, they still have relatively low drug load and biodistribution, leading to high uptake in the liver and spleen, thus risking acute cumulative drug injury.

Accordingly, there is still a need in the art for nanoparticles that can provide one or more of—better drug load capability, targeted uptake, and enhanced transfection efficiency, and/or which can be consistently manufactured cost-effectively on a large scale.

The present disclosure aims to meet one or more of these needs, provide other benefits, or at least provide the public with a useful choice. Provided herein are lipid-coated nanoparticles (LC-NPs), such as lipid-coated lipid nanoparticles (LC-LNPs), which in some embodiments have enhanced transfection efficiency and/or a scalable and/or CMC friendly manufacturing process.

In an aspect, provided herein are lipid-coated nanoparticles comprising:
(a) a nanoparticle;
(b) a plurality of payload molecules entrapped in the nanoparticle;
(c) a lipid coating around the nanoparticle and the plurality of payload molecules. To be clear, the lipid coating is a separate element of the lipid-coated nanoparticle in addition to the nanoparticle (around which the lipid coating is located).

In an aspect, provided herein are pharmaceutical compositions comprising the lipid-coated nanoparticle disclosed herein, and a pharmaceutically acceptable excipient.

In another aspect, provided herein is a method for manufacturing lipid-coated lipid nanoparticles (LNPs) comprising:
(a) dissolving at least one payload molecule into a first solution or a second solution, wherein the first solution comprises an aqueous phase and the second solution comprises an organic phase and a plurality of molecules capable of self-assembly, and wherein the first and second solutions are miscible;
(b) mixing the first solution and the second solution using microfluidics to obtain lipid nanoparticles encapsulating the at least one payload molecule under conditions suitable for LNP formation, thereby forming LNPs;
(c) purifying said LNPs;
(d) adjusting LNP concentration in an aqueous phase; and
(e) mixing said LNPs and a third solution using microfluidics to obtain lipid-coated LNPs, wherein the third solution comprises an organic phase and a plurality of molecules capable of self-assembly, and the third solution contains the same or different molecules as the second solution.

In another aspect, provided herein is a method for manufacturing lipid-coated lipid nanoparticles (LNPs) comprising mixing LNPs with a third solution using microfluidics to obtain lipid-coated LNPs, wherein:
the LNPs were formed by mixing a first solution and a second solution using microfluidics under conditions suitable for LNP formation to obtain lipid nanoparticles encapsulating at least one payload molecule;
the first solution comprises an aqueous phase and the second solution comprises an organic phase and a plurality of molecules capable of self-assembly;
the first and second solutions are miscible;
the at least one payload molecule was dissolved in the first solution or the second solution;
the third solution comprises an organic phase and a plurality of molecules capable of self-assembly, and the third solution contains the same or different molecules as the second solution.

In yet another aspect, provided herein is a method for manufacturing lipid-coated lipid nanoparticles (LNPs) comprising:
(a) preparing a first solution comprising an aqueous phase;
(b) preparing a second solution comprising an organic phase and a plurality of molecules capable of self-assembly, and wherein the first and second solutions are miscible;
(c) dissolving at least one payload molecule into the first or second solution;
(d) mixing said first and second solutions using microfluidics to obtain lipid nanoparticles encapsulating said payload under conditions suitable for LNP formation;
(e) purifying said LNPs;
(f) adjusting LNP concentration in aqueous phase;
(g) preparing a third solution comprising an organic phase and a plurality of molecules capable of self-assembly, wherein the third solution contains the same or different molecules as the second solution; and
(h) mixing said LNPs and third solution using microfluidics to obtain lipid-coated LNPs.

In embodiments, the lipid-coated lipid nanoparticles (LNPs) are purified after the manufacturing process.

In an aspect, provided herein is a method for transfecting a cell comprising contacting the cell with a lipid-coated nanoparticle as described herein or a pharmaceutical composition as described herein, including in embodiments.

In an aspect, provided herein is a method for administering a payload to a subject, comprising administering a lipid-coated nanoparticle as described herein or a pharmaceutical composition as described herein, including in embodiments, to a subject in need thereof.

### BRIEF DESCRIPTION OF THE DRAWINGS

FIG. 1 depicts schematic representation of liposome, lipid micelle, lipid nanoparticle (LNP), and lipid-coated lipid nanoparticle (LC-LNP) (inside a liposome or inside a micelle).

FIG. 2 depicts schematic representation of a functionalized liposome and a functionalized liposome encapsulating a lipid nanoparticle (LNP).

FIG. 3 depicts schematic representation of a micelle, a liposome, a nanoemulsion, and a solid lipid nanoparticle (SLN).

FIG. 4 shows a high throughput workflow for producing lipid-coated LNPs (LC-LNPs).

FIG. 5A shows a particle size distribution graph, after manual mixing of liposomes and LNPs, immediately upon mixing (T₀) and after 24 hours (T₂₄).

FIG. 5B shows a particle size distribution graph, after microfluidic mixing of lipid solution and LNPs, immediately upon mixing (T₀) and after 24 hours (T₂₄).

FIG. 6 shows a particle size distribution graph, after manual mixing of liposomes and emulsion nanoparticles, immediately upon mixing (T₀) and after 72 hours (T₇₂).

FIG. 6B shows a particle size distribution graph, after microfluidic mixing of lipid solution and emulsion nanoparticles, immediately upon mixing (T₀) and after 1 hour (T₁).

FIGS. 7A-B are scatterplots showing transfection efficiency in Hek 293 cells. LPX-U-GFP-LNP0607 (FIG. 7A) and positive control Lipofectamine Messenger MAX (FIG. 7B).

FIGS. 8A-B are scatterplots showing transfection efficiency in P6 Jurkat cells. LPX-U-GFP-LNP0607 (FIG. 8A) and positive control Lipofectamine Messenger MAX (FIG. 8B).

FIGS. 9A-D are scatterplots showing transfection efficiency in primary T cells. BAE-LPX-U-GFP (KT001)-LNP (FIG. 9A), positive control Lipofectamine Messenger MAX (FIG. 9B), lipid coating alone (liposome-BAE) (FIG. 9C), and the LNP alone (LNP-STI-KT001) (FIG. 9D).

FIGS. 10A-B are scatterplots showing transfection efficiency in primary T cells. KT-001-LPX-U-GFP (KT001)-LNP (FIG. 10A) and GFP (KT001)-LNP (FIG. 10B).

FIGS. 11A-D show Luc expression levels of rsF548 and rsF549 following i.m. injection in mice. Total Luc expression levels (FIG. 11A), Luc expression levels in muscle (FIG. 11B), Luc expression levels in liver (FIG. 11C), and relative Luc expression levels liver: muscle (FIG. 11D).

### DETAILED DESCRIPTION OF CERTAIN EMBODIMENTS

### Definitions

Unless defined otherwise, technical and scientific terms used herein have meanings that are commonly understood by those of ordinary skill in the art unless defined otherwise. Generally, terminologies pertaining to techniques of cell and tissue culture, molecular biology, immunology, microbiology, genetics, transgenic cell production, protein chemistry and nucleic acid chemistry and hybridization described herein are well known and commonly used in the art. Methods and techniques pertaining to techniques of cell and tissue culture, molecular biology, immunology, microbiology, genetics, transgenic cell production, protein chemistry and nucleic acid chemistry and hybridization are generally performed according to conventional procedures well known in the art and as described in various general and more specific references that are cited and discussed herein unless otherwise indicated. See, e.g., Sambrook et al. Molecular Cloning: A Laboratory Manual, 2d ed., Cold Spring Harbor Laboratory Press, Cold Spring Harbor, N.Y. (1989) and Ausubel et al., Current Protocols in Molecular Biology, Greene Publishing Associates (1992). A number of basic texts describe standard antibody production processes, including, Borrebaeck (ed) Antibody Engineering, 2nd Edition Freeman and Company, NY, 1995; McCafferty et al. Antibody Engineering, A Practical Approach IRL at Oxford Press, Oxford, England, 1996; and Paul (1995) Antibody Engineering Protocols Humana Press, Towata, N.J., 1995; Paul (ed.), Fundamental Immunology, Raven Press, N.Y, 1993; Coligan (1991) Current Protocols in Immunology Wiley/Greene, NY; Harlow and Lane (1989) Antibodies: A Laboratory Manual Cold Spring Harbor Press, NY; Stites et al. (eds.) Basic and Clinical Immunology (4th ed.) Lange Medical Publications, Los Altos, Calif., and references cited therein; Coding Monoclonal Antibodies: Principles and Practice (2nd ed.) Academic Press, New York, N.Y., 1986, and Kohler and Milstein Nature 256:495-497, 1975. All of the references cited herein are incorporated herein by reference in their entireties. Enzymatic reactions and enrichment/purification techniques are also well known and are performed according to manufacturer's specifications, as commonly accomplished in the art or as described herein. The terminology used in connection with, and the laboratory procedures and techniques of, analytical chemistry, synthetic organic chemistry, and medicinal and pharmaceutical chemistry described herein are well known and commonly used in the art. Standard techniques can be used for chemical syntheses, chemical analyses, pharmaceutical preparation, formulation, and delivery, and treatment of patients.

The section headings used herein are for organizational purposes only and are not to be construed as limiting the desired subject matter in any way. In the event that any literature incorporated by reference contradicts any term defined in this specification, this specification controls. While the present teachings are described in conjunction with various embodiments, it is not intended that the present teachings be limited to such embodiments. On the contrary, the present teachings encompass various alternatives, modifications, and equivalents, as will be appreciated by those of skill in the art. The features of any embodiment described herein can be combined with the features of any one or more other embodiments described herein, provided that the embodiments are not inconsistent with each other.

Before describing the present teachings in detail, it is to be understood that the disclosure is not limited to specific compositions or process steps, as such may vary. It should be noted that, as used in this specification and the appended claims, the singular form “a”, “an” and “the” include plural references unless the context clearly dictates otherwise. Thus, for example, reference to “a conjugate” includes a plurality of conjugates and reference to “a cell” includes a plurality of cells and the like.

It is understood the use of the alternative (e.g., “or”) herein is taken to mean either one or both or any combination thereof of the alternatives.

The term “and/or” used herein is to be taken to mean specific disclosure of each of the specified features or components with or without the other. For example, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As used herein, the term “about” refers to a value or composition that is within an acceptable error range for the particular value or composition as determined by one of ordinary skill in the art, which will depend in part on how the value or composition is measured or determined, i.e., the limitations of the measurement system. For example, “about” or “approximately” can mean within one or more than one standard deviation per the practice in the art. Alternatively, “about” or “approximately” can mean a range of up to 10% (i.e., +10%) or more depending on the limitations of the measurement system. For example, about 5 mg can include any number between 4.5 mg and 5.5 mg. Furthermore, particularly with respect to biological systems or processes, the terms can mean up to an order of magnitude or up to 5-fold of a value. When particular values or compositions are provided in the instant disclosure, unless otherwise stated, the meaning of “about” or “approximately” should be assumed to be within an acceptable error range for that particular value or composition. In some embodiments, “about” encompasses variation within 10%, 5%, 2%, 1%, or 0.5% of a stated value.

Numeric ranges are inclusive of the numbers defining the range. Measured and measurable values are understood to be approximate, taking into account significant digits and the error associated with the measurement. Also, all ranges are to be interpreted as encompassing the endpoints in the absence of express exclusions such as “not including the endpoints”; thus, for example, “ranging from 1 to 10” includes the values 1 and 10 and all integer and (where appropriate) non-integer values greater than 1 and less than 10.

The use of “comprise”, “comprises”, “comprising”, “contain”, “contains”, “containing”, “include”, “includes”, and “including” and their grammatical variants, as used herein are intended to be non-limiting so that one item or multiple items in a list do not exclude other items that can be substituted or added to the listed items. It is to be understood that both the foregoing general description and detailed description are exemplary and explanatory only and are not restrictive of the teachings. Unless specifically noted in the above specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims).

The terms “effective amount”, “therapeutically effective amount” or “effective dose” or related terms may be used interchangeably and refer to an amount of the therapeutic agent that when administered to a subject, is sufficient to affect a measurable improvement or prevention of a disease or disorder associated with, for example, coronavirus infection. For example, administering an effective dose sufficient to inhibit the proliferation and/or replication of the coronavirus, and/or the development of the viral infection within the subject. Therapeutically effective amounts of the therapeutic agents provided herein, when used alone or in combination with another drug, will vary depending upon the relative activity of the therapeutic agent, and depending upon the subject and disease condition being treated, the weight and age and sex of the subject, the severity of the disease condition in the subject, the manner of administration and the like, which can readily be determined by one of ordinary skill in the art. In one embodiment, a therapeutically effective amount will depend on certain aspects of the subject to be treated and the disorder to be treated and may be ascertained by one skilled in the art using known techniques. In addition, as is known in the art, adjustments for age as well as the body weight, general health, sex, diet, time of administration, drug interaction, and the severity of the disease may be necessary.

The terms “subject” and “patient” as used herein refer to human and non-human animals, including vertebrates, mammals and non-mammals. In one embodiment, the subject can be a human, a non-human primate, simian, ape, murine (e.g., mice and rats), bovine, porcine, equine, canine, feline, caprine, lupine, ranine or piscine.

The terms “administering”, “administered”, and grammatical variants thereof refer to the physical introduction of a therapeutic agent to a subject, using any of the various methods and delivery systems known to those skilled in the art. Exemplary routes of administration for the formulations disclosed herein include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In one embodiment, the formulation is administered via a non-parenteral route, e.g., orally. Other non-parenteral routes include a topical, epidermal or mucosal route of administration, for example, intranasally, vaginally, rectally, sublingually or topically. Administering can also be performed, for example, once, a plurality of times, and/or over one or more extended periods.

“Treating” is to be understood broadly and encompasses any beneficial effect, including, e.g., delaying, slowing, or arresting the worsening of symptoms associated with pulmonary inflammatory disease or remedying such symptoms, at least in part. Treating also encompasses bringing about any form of improved patient function, as discussed in detail below. In some embodiments, treatment also means prolonging survival as compared to expected survival if not receiving treatment. Those in need of treatment include those who already have the disease or disorder, as well as those who tend to have the disease or disorder or who should prevent the disease or disorder. In embodiments, the terms “treatment” and “treating” refer to fighting the coronavirus infection in a human or animal subject. By virtue of the administration of at least one embodiment of the compositions described herein, the viral infection rate (infectious titer) in the subject will decrease, and the virus may completely disappear from the subject. The terms “treatment” and “treating” also refers to attenuating symptoms associated with the viral infection (e.g., respiratory syndrome, kidney failure, fever, and other symptoms relating to coronavirus infections).

The term “synergistic effect” refers to a situation where the combination of two or more agents produces a greater effect than the sum of the effects of each of the individual agents. The term encompasses not only a reduction in symptoms of the disorder to be treated, but also an improved side effect profile, improved tolerability, improved patient compliance, improved efficacy, or any other improved clinical outcome.

The term a “sub-therapeutic amount” of an agent or therapy is an amount less than the effective amount for that agent or therapy as a single agent, but when combined with an effective or sub-therapeutic amount of another agent or therapy can produce a result desired by the physician, due to, for example, synergy in the resulting efficacious effects, or reduced side effects.

Combination therapy or “in combination with” refers to the use of more than one therapeutic agent to treat a particular disorder or condition. By “in combination with,” it is not intended to imply that the therapeutic agents must be administered at the same time and/or formulated for delivery together, although these methods of delivery are within the scope of this disclosure. A therapeutic agent can be administered concurrently with, prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks before), or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks after), one or more other additional agents. The therapeutic agents in a combination therapy can also be administered on an alternating dosing schedule, with or without a resting period (e.g., no therapeutic agent is administered on certain days of the schedule). The administration of a therapeutic agent “in combination with” another therapeutic agent includes, but is not limited to, sequential administration and concomitant administration of the two agents. In general, each therapeutic agent is administered at a dose and/or on a time schedule determined for that particular agent.

The terms “nucleic acid”, “polynucleotide” and “oligonucleotide” and other related terms used herein are used interchangeably and refer to polymers of nucleotides and are not limited to any particular length. The term refers to a polymer containing at least two deoxyribonucleotides or ribonucleotides in either single- or double-stranded form and includes DNA, RNA, and hybrids thereof. Nucleic acids include recombinant and chemically-synthesized forms. Nucleic acids include DNA molecules (cDNA or genomic DNA), RNA molecules (e.g., mRNA, siRNA, dsRNA, shRNA, miRNA, tRNA, rRNA, vRNA), analogs of the DNA or RNA generated using nucleotide analogs (e.g., peptide nucleic acids and non-naturally occurring nucleotide analogs), and hybrids thereof. Nucleic acid molecules can be single-stranded or double-stranded. In some embodiments, the nucleic acid molecules of the disclosure comprise a contiguous open reading frame encoding an Fc-coronavirus antigen fusion protein, or a derivative, mutein, or variant thereof. In one embodiment, nucleic acids comprise one type of polynucleotides or a mixture of two or more different types of polynucleotides.

The terms “lipid” or “lipid moiety” are used in accordance with its ordinary meaning in chemistry and refer to a hydrophobic molecule which is typically characterized by an aliphatic hydrocarbon chain. In embodiments, the lipid moiety includes a carbon chain of 3 to 100 carbons. In embodiments, the lipid moiety includes a carbon chain of 5 to 50 carbons. In embodiments, the lipid moiety includes a carbon chain of 5 to 25 carbons. In embodiments, the lipid moiety includes a carbon chain of 8 to 25 carbons. Lipid moieties may include saturated or unsaturated carbon chains, and may be optionally substituted. In embodiments, the lipid moiety is optionally substituted with a charged moiety at the terminal end. In embodiments, the lipid moiety is an alkyl or heteroalkyl optionally substituted with a carboxylic acid moiety at the terminal end. The term “lipid” also refers to a group of organic compounds that include, but are not limited to, esters of fatty acids and are characterized by being insoluble in water, but soluble in many organic solvents. They can be divided into several broad classes: (1) “simple lipids,” which include fats and oils as well as waxes; (2) “compound lipids,” which include phospholipids and glycolipids; and (3) “derived lipids” such as steroids. Lipids can also be subdivided into subgroups based on their charges and/or specific purpose. For example, lipids can be anionic, cationic, neutral, zwitterionic, ionizable cationic, and general “helper lipids”.

The term “ionizable cationic lipid” refers to lipids that are protonated (e.g., >50% protonated) at low pH (e.g., pH 4), which makes them positively charged, but they may remain neutral at physiological pH (e.g., pH 7.4). In embodiments, the ionizable cationic lipids include, but are not limited to, ALC-0315 [(4-hydroxybutyl)azanediyl]di(hexane-6,1-diyl)bis(2-hexyldecanoate) and its analogs, SM-102 (1-octylnonyl 8-[(hydroxyethyl)[6-oxo-6-(undecyloxy)hexyl]amino]octanoate) and its analogs, DODMA (1,2-dioleyloxy-3-dimethylaminopropane), DODAP (1,2-dioleoyl-3-trimethylammonium propane), KT001 (also referred to as KT-001, ionizable cationic lipid (proprietary-U.S. provisional 63/313,648)), and BAE (ionizable cationic lipid (proprietary-U.S. provisional 63/313,648)).

The term “cationic lipid” refers to any number of lipid species that have a net positive charge. The term “anionic lipid” refers to any number of lipid species that have a net negative charge.

The term “helper lipid” refers to lipids that improve nanoparticle stability, fluidity, blood compatibility, oligonucleotide delivery efficiency, and transfection activity. In embodiments, the helper lipids include, but is not limited to, some phospholipids, DOPE (1,2-dioleoyl-sn-glycero-3-phosphoethanolamine), DOPC (1,2-dioleoyl-sn-glycero-3-phosphocholine), ALC-0159 (2-[(polyethylene glycol)-2000]-N,N-ditetradecylacetamide), DEPE (1,2-dielaidoyl-sn-phosphatidylethanolamine), DLOPE (1,2-dilinoleoyl-sn-glycero-3-phosphoethanolamine), POPE (1-palmitoyl-2-oleoyl-sn-glycero-3-phosphoethanolamine), DSPC (distearoylphosphatidylcholine), cholesterol, cholesterol-based lipids, and PEGylated lipids.

The term “lipid conjugate” refers to a conjugated lipid that inhibits aggregation of lipid particles. Such lipid conjugates include, but are not limited to, PEG (polyethylene glycol)-lipid conjugates such as, e.g., PEG coupled to dimyristoylglycerols (e.g., PEG-DMG conjugates), PEG coupled to diacylglycerols (e.g., PEG-DAG conjugates), PEG coupled to dialkyloxypropyls (PEG-DAA), PEG coupled to cholesterol, PEG coupled to phosphatidylethanolamines (e.g., phosphatidylethanolamine (PEG-PE), and PEG conjugated to ceramides (e.g., mPEG2000-1,2-di-O-alkyl-sn3-carbomoylglyceride (PEG-C-DOMG), 1-[8′-(1,2-dimyristoyl-3-propanoxy)-carboxamido-3′,6′-dioxaoctanyl]carbamoyl-co-methyl-poly(ethylene glycol) (2 KPEG-DMG), and 1,2-Dimyristoyl-rac-glycero-3-methylpolyoxyethylene (DMG-PEG)). Other types of lipid conjugates are POZ (poly(2-oxazoline)-lipid conjugates and ATTA (14′-amino-3′,6′,9′,12′-tetraoxatetradecanoic acid))-lipid conjugates.

The term “nanoparticle” refers to submicron-sized colloidal particles. In general, such nanoparticles will have a particle size of at least 10 nm, but less than 1,000 nm, preferably less than 500 nm, and more preferably less than about 150 nm. In embodiments, the nanoparticles are less than about 100 nm, less than about 50 nm, less than about 20 nm. In embodiments, the nanoparticles are between about 50 nm and 180 nm. In embodiments, the nanoparticles are between about 50 nm and 150 nm. In embodiments, the nanoparticles are between about 70 nm and 130 nm. Nanoparticles may be composed of any appropriate material. In embodiments, the nanoparticles are lipid-based nanoparticles. In embodiments, the nanoparticles are inorganic nanoparticles. In embodiments, the nanoparticles are polymeric nanoparticles. In embodiments, the nanoparticle has the shape of a sphere, rod, cube, triangular, hexagonal, cylinder, spherocylinder, or ellipsoid.

The term “lipid-based nanoparticle” refers to nanoparticles composed of lipids, including but not limited to, lipid nanoparticles (LNPs), solid lipid nanoparticles (SLNs), nanoemulsion and the like. These nanoparticles include a lipid formulation that can be used to deliver an active agent or therapeutic agent, such as a nucleic acid (e.g., an mRNA), to a target site of interest (e.g., cell, tissue, organ, and the like). In embodiments, the active agent or therapeutic agent, such as a nucleic acid, may be encapsulated in the lipid-based nanoparticle, thereby protecting it from enzymatic degradation.

As used herein, the term “self-assembling molecule”, refers to any molecule capable of a defined arrangement without guidance or management from an outside source. The optimized LNPs may be comprised of a single species of self-assembling molecule or may be comprised of a plurality of species of self-assembling molecule. In various embodiments, the LNPs include a lipid-component with at least one species of lipid molecule. In embodiments, the LNP may contain a polymer molecule and/or a protein/peptide molecule. In embodiments, the self-assembling molecules of the LNP may only include lipid molecules. The lipid component may comprise a single lipid species, or it may include more than one lipid species. In embodiments, the relative composition of lipid in a LNP preparation will be varied.%

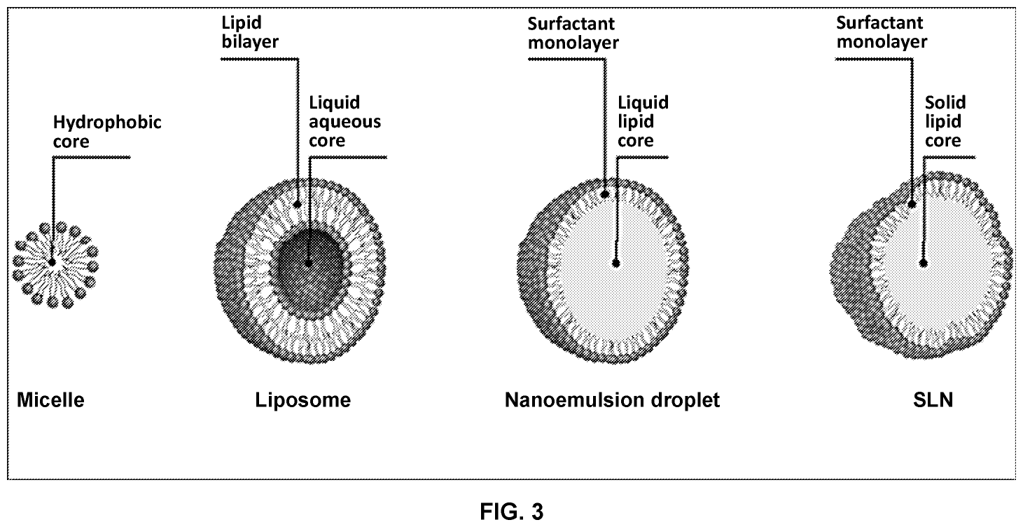

The term “liposome” refers to a structure (vesicle) composed of one or more lipid bilayers and an aqueous core (illustrated in FIG. 3). Liposomes are classified by lamellarity and size. Liposomes vary in size from about 50 nm to about 3000 nm. Liposomes can deliver hydrophobic cargo in their bilayer and/or hydrophilic cargo in their aqueous core.

The term “micelle” refers to self-assemblies of lipid monolayers in aqueous solutions. Micelles are vesicles that have a hydrophobic core, where the phospholipid (or non-polar) tails are oriented towards the interior, and can be used to encapsulate hydrophobic cargo (in FIG. 3). Unlike in SLNs or nanoemulsion, no oil is trapped in the core. Micelles can vary in size from about 2 nm to about 100 nm.

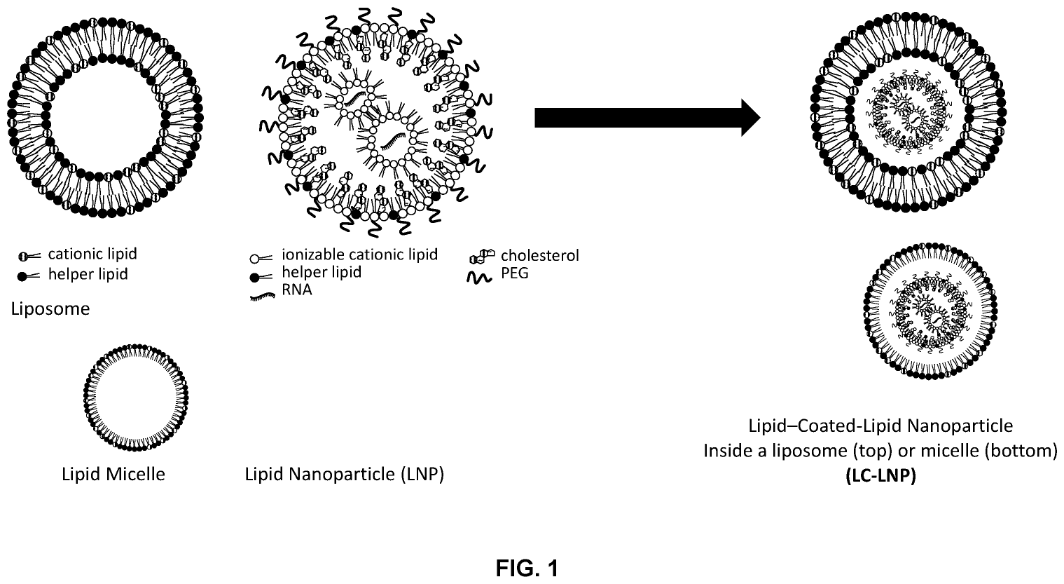

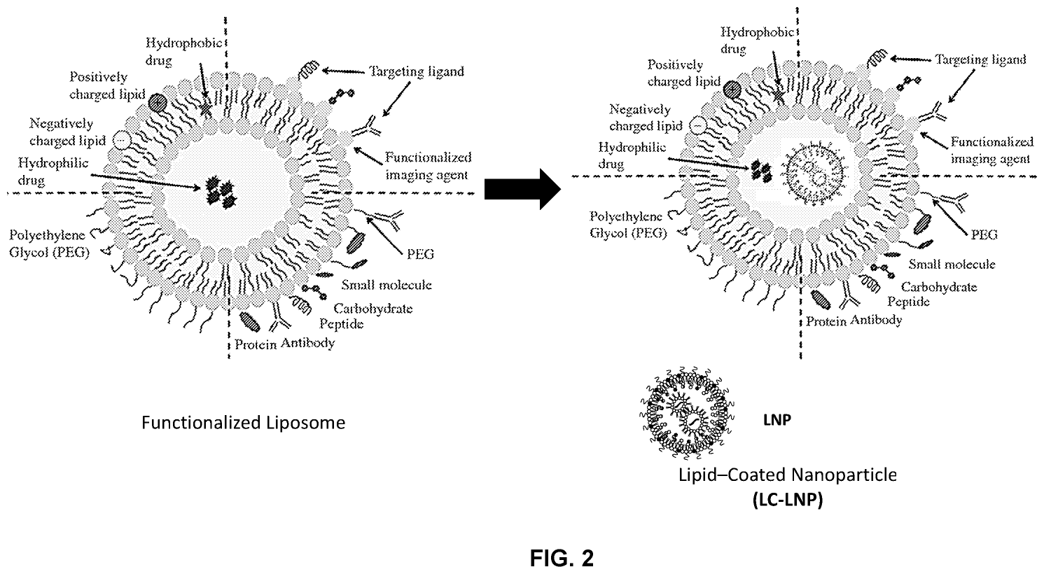

The term “lipid nanoparticle” or “LNP” refer to a lipid structure where a lipid shell surrounds an internal core which encapsulates reverse micelles each of which encapsulates hydrophilic cargo, as shown in FIG. 1 and FIG. 2. LNPs can vary in size from about 20 nm to about 100 nm. The hydrophilic cargo such as, for example, oligonucleotide, peptide, RNA, DNA, mRNA, pDNA.

The term “reverse micelle” refers to a structure that is inverted compared with the traditional micelle. Reverse micelles form a hydrophilic core, with the phospholipid (or non-polar) tails oriented towards the exterior, and can be used to encapsulate small hydrophilic cargo, similarly to LNPs.

The term “solid lipid nanoparticle” or “SLN” refers to a lipid-based nanoparticle having a surfactant shell (which is a type of lipid, for example, one or more phospholipids such as phosphatidylcholine) surrounding a core matrix composed of solid lipids (illustrated in FIG. 3). Solid lipids, known as fats, are solid at ambient temperature, are typically saturated and include, for example, glycerides, waxes, and fatty acids. SLNs can vary in size from about 40 nm to about 1000 nm. SLNs are used to encapsulate hydrophobic and/or hydrophilic cargo. Examples of lipid surfactants, include but are not limited to, TPGS (D-α-tocopherol polyethylene glycol succinate) 1000; polysorbate 80; sorbitan monolaurate, sorbitan monooleate, and the like, polyoxyethylene hydrogenated castor oil 60, polyoxyethylene lauryl alcohol and the like; glycerol fatty acid ester and the like; phospholipids such as phosphatidylcholine; sodium dodecyl sulfate, sodium lauryl sulfate, sodium cholate, sodium deoxycholate, sodium taurodeoxycholate and the like. Despite their name, “solid lipid nanoparticles” are not lipid nanoparticles (LNPs) as defined above in paragraph [0052], but rather are a different type of lipid-based nanoparticles as defined above in paragraph [0048].

The term “emulsion nanoparticle,” “emulsion lipid nanoparticle,” or “nanoemulsion” are used interchangeably and refer to a nanoparticle system made by adding oil to drug solution diluted in organic solvent, e.g., ethanol, and stirring until the organic solvent evaporates (illustrated in FIG. 3). The nanoparticles thus formed contain a liquid lipid hydrophobic core surrounded by a surfactant shell (which is a type of lipid, for example, phospholipids such as phosphatidylcholine). Despite their name, an “emulsion lipid nanoparticle” is not a lipid nanoparticle (LNP) as defined above in paragraph [0052], but rather is a different type of lipid-based nanoparticle as defined above in paragraph [0048].

As used herein, “N/P ratio” refers to the ratio of positively chargeable polymer amine (N=nitrogen) groups (in lipid) to negatively-charged nucleic acid phosphate (P) groups (in payload). The N/P ratio plays an important role in intracellular payload delivery. In various embodiments, the payload's N:P ratio is varied. In various embodiments, the N:P ratio is varied between about 0.5 to about 5. In various embodiments, the N:P ratio is varied between about 0.25 and about 10. In various embodiments, the N:P ratio is about 0.1, about 0.2, about 0.25, about 0.5, about 1, about 1.5, about 2, about 2.5, about 3, about 3.5, about 4, about 4.5, about 5, about 6, about 7, about 8 about 9, or about 10.

The term “derivative” refers to a compound that can be derived from a similar compound (i.e., a so-called “reference” compound) by a chemical reaction.

“Analog,” or “analogue” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

The terms “Pharmaceutically acceptable excipient” and “pharmaceutically acceptable carrier” refer to a substance that aids the administration of an active agent to and absorption by a subject and can be included in the compositions of the present disclosure without causing a significant adverse toxicological effect on the patient. Non-limiting examples of pharmaceutically acceptable excipients contemplated herein include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful in the present disclosure.

As used herein, the term “salt” refers to acid or base salts of the compounds used in the methods of the present invention. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

The term “pharmaceutically acceptable salts” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Thus, the compounds of the present disclosure may exist as salts, such as with pharmaceutically acceptable acids. The present disclosure includes such salts. Non-limiting examples of such salts include hydrochlorides, hydrobromides, phosphates, sulfates, methanesulfonates, nitrates, maleates, acetates, citrates, fumarates, proprionates, tartrates (e.g., (+)-tartrates, (−)-tartrates, or mixtures thereof including racemic mixtures), succinates, benzoates, and salts with amino acids such as glutamic acid, and quaternary ammonium salts (e.g. methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents.

The abbreviations used herein have their conventional meaning within the chemical and biological arts. The chemical structures and formulae set forth herein are constructed according to the standard rules of chemical valency known in the chemical arts. Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

### Overview

Liposomes are attractive as gene vectors due to their ability to carry DNA to various target cells. In addition, liposome formulations have been established to be a safe carrier, with such formulations being used worldwide in different therapeutic and vaccinology products. Liposomes have also been used as a drug carrier to control drug delivery to protect the drug payload from rapid degradation, to enhance drug concentration in targeted tissues and to lower doses of the required drug and, hence lowering toxicity. The versatile structure and low immunogenicity of liposomes have been shown to be a promising gene transfer system. Liposomes can entrap different molecules such as nucleic acids and may even protect DNA against enzymatic degradation within the cell. Liposomes also can enhance cellular uptake, endosomal escape, and gene transfection. However, their application in gene therapy is hampered by low transfection efficiency. Lipid-based nanoparticles have been found to have better transfection efficiencies than liposomes and have been replacing liposomes as drug delivery systems. However, there is a lack of streamlined methods for lipid nanoparticle (LNP) manufacturing.

Lipid nanoparticle (LNP) manufacturing for drug delivery is challenging due to their complicated physicochemical properties that are affected by various formulation parameters. Controlling for particle structure and size distribution, physicochemical properties of the particle surface, lipid content, amount of the free API and encapsulation efficiency, and physical and chemical stability in LNP manufacture is difficult and complicated. LNPs have low drug load and biodistribution, leading to high uptake in the liver and spleen, thus risking acute cumulative drug injury. Additionally, transfection efficiency of LNPs although better than that of liposomes, is not very high and hard to control during manufacturing. Similar issues exist in the manufacturing of nanoemulsions and solid lipid nanoparticles for drug delivery.

Controlling particle structure and size distribution, physicochemical properties of the particle surface, lipid content, and physical and chemical stability of liposomes and micelles is much simpler than controlling these same properties in LNPs. Unexpectedly, when lipid-based nanoparticles (in aqueous solution) were mixed with lipids (in organic solution) the nanoparticles remained intact (facilitated by controlling the ratio of aqueous buffer to organic solution, as described herein), yielding lipid-coated lipid-based nanoparticles (such as LC-LNPs, LC-nanoemulsion, and LC-SLNs). Surprisingly, these lipid-coated lipid-based nanoparticles have significantly improved transfection efficiencies compared to liposomes alone, nanoemulsions alone, or LNPs alone

### Lipid-Coated Nanoparticle Compositions

In an aspect, provided herein are lipid-coated nanoparticles comprising:
(a) a nanoparticle;
(b) a plurality of payload molecules entrapped in the nanoparticle;
(c) a lipid coating around the nanoparticle and the plurality of payload molecules. To be clear, the lipid coating is a separate element of the lipid-coated nanoparticle in addition to the nanoparticle (around which the lipid coating is located).

In embodiments, the lipid-coated nanoparticle comprises a nanoparticle, with a plurality of payload molecules entrapped in the nanoparticle, surrounded by a lipid shell such as a lipid bilayer, thus forming a liposome containing the nanoparticle. In embodiments, the lipid-coated nanoparticle comprises a nanoparticle, with a plurality of payload molecules entrapped in the nanoparticle, surrounded by a lipid shell such as a lipid monolayer, thus forming a lipid micelle containing the nanoparticle.

In embodiments, the lipid coating surrounding the nanoparticle comprises at least one species of lipid molecule. In embodiments, the lipid coating surrounding the nanoparticle comprises one or more ionizable cationic lipid species, one or more cationic lipid species, one or more anionic lipid species, one or more neutral lipid species, or one or more helper lipid species. In embodiments, the lipid coating comprises an ionizable cationic lipid species, a cationic lipid species, an anionic lipid species, a neutral lipid species, a helper lipid species, or any combination thereof.

In embodiments, the lipid coating surrounding the nanoparticle comprises at least one ionizable cationic lipid species. In embodiments, the lipid coating surrounding the nanoparticle comprises at least one cationic lipid species. In embodiments, the lipid coating surrounding the nanoparticle comprises at least one anionic lipid species. In embodiments, the lipid coating surrounding the nanoparticle comprises at least one neutral lipid species. In embodiments, the lipid coating surrounding the nanoparticle comprises at least one helper lipid species.

In embodiments, the lipid coating comprises one type or species of lipid. In embodiments, the lipid coating comprises more than one type of lipid. In embodiments, the lipid coating comprises at least two types of lipids. In embodiments, the lipid coating comprises at least three types of lipids. In embodiments, the lipid coating comprises at least four types of lipids. In embodiments, the lipid coating comprises at least five types of lipids. In embodiments, the lipid coating comprises at least six types of lipids. In embodiments, the lipid coating comprises at least seven types of lipids.

In embodiments, the lipid coating composition comprises ionizable cationic lipid species. Such lipids include, but are not limited to, ALC-0315 and its analogs, SM-102 and its analogs, 7-[(2-Hydroxyethyl) [8-(nonyloxy)-8-oxooctyl]amino]heptyl 2-octyldecanoate, 98N12-5, 9A1P9, D-Lin-MC3-DMA, A6, OF-02, A18-Iso5-2DC18, L319, DODMA (1,2-dioleyloxy-3-dimethylaminopropane), DODAP (1,2-dioleoyl-3-trimethylammonium propane), DLin-KC2-DMA, C12-200, 3060i10, BP-Lipid 215, bis(N-2-ethoxyethyl 2-hexyldecanoate)amine, BP-Lipid 400, BP-Lipid 600, BP-Lipid 700, BP-Lipid 800, BP-Lipid 1000, C12-200, cKK-E12, 7C1, GO-G14, L319, 304O₁₃, 306-O12B, OF-Deg-Lin, 306Oᵢ₁₀, FTT5, KT001, and BAE.

In embodiments, the lipid coating composition comprises cationic lipid species. Such lipids include, but are not limited to, DODAC (dioctadecyldimethylammonium chloride), DBOP (dibutyl 2-octylamino-2-propanephosphonate), DBPP (N,N¹-dimethyl-N,N¹-bis[2-(oleoyloxy)ethyl]piperazine), MeBOP (N-methyl-N-bisp-(oleoyloxy)ethyl]piperazine), DBOP (N,N¹-dimethyl-N,N¹-bis[2-(palmitoyloxy)ethyl]piperazine), EDOPC (2-[2,3-bis[[(Z)-octadec-9-enoyl]oxy]propoxy-ethoxyphosphoryl]oxyethyl-trimethylazanium), TAP (1,2-dioleoyl-3-trimethylammonium-propane), PolyGum, DOTMA (1,2-di-O-octadecenyl-3-trimethylammonium propane), DDAB (Dimethyldioctadecylammonium bromide), DOTAP (1,2-dioleoyl-3-trimethylammonium propane), DC-Chol, DLRIE (2,3-bis(dodecyloxy)-N-(2-hydroxyethyl)-N,N-dimethylpropan-1-amonium bromide), GAP-DLRIE ((±)-N-(3-aminopropyl)-N,N-dimethyl-2,3-bis(dodecyloxy)-1-propanaminium bromide), distearylamine, vectamidine, DOGS (dioctadecylamidoglycylspermine), DOSPA (2,3-dioleyloxy-N-[2 (sperminecarboxamido)ethyl]-N,N-dimethyl-1-propanaminium), dipalmitylamine, dimyristylamine, EDMPC (2-(((2,3-bis(tetradecanoyloxy)propoxy)(ethoxy)phosphoryl)oxy)-N,N,N-trimethylethan-1-aminium trifluoromethanesulfonate), DOSPER (1,3-di-oleoyloxy-2-(6-carboxy-spermyl)-propylamid), EDLPC (1-0,2-O-Dilauroyl-3-O-[2-(trimethylaminio) ethoxyethoxyphosphinyl]-L-glycerol), DORIE (N-(2-hydroxyethyl)-N,N-dimethyl-2,3-bis(((Z)-octadec-9-en-1-yl)oxy) propan-1-aminium bromide), DMOBA (N,N-dimethyl-3,4-dioleyloxybenzylamine), DDAB (N,N-distearyl-N,N-dirnethylarnrnonium bromide), and DMRIE (N-(1,2-dimyristyloxyprop-3-yl)-N,N-dimethyl-N-hydroxyethyl ammonium bromide).

In embodiments, the lipid coating composition comprises helper lipid species. Such lipids include, but are not limited to, DOPE (1,2-dioleoyl-sn-glycero-3-phosphoethanolamine), DOPC (1,2-dioleoyl-sn-glycero-3-phosphocholine), ALC-0159 (2-[(polyethylene glycol)-2000]-N,N-ditetradecylacetamide), DEPE (dielaidoyl-phosphatidylethanolamine), DLOPE (1,2-dilinoleoyl-sn-glycero-3-phosphoethanolamine), POPE (palmitoyloleoyl-phosphatidylethanolamine), DSPC (1,2-distearoyl-sn-glycero-3-phosphocholine), sterols (e.g., cholesterol), cholesterol-based lipids, and PEGylated lipids.

In embodiments, helper lipids may be sterols such as cholesterol and derivatives thereof. Non-limiting examples of cholesterol derivatives include polar analogues such as 5α-cholestanol, 5β-coprostanol, cholesteryl-(2′-hydroxy)-ethyl ether, cholesteryl-(4′-hydroxy)-butyl ether, and 6-ketocholestanol; non-polar analogues such as 5α-cholestane, cholestenone, 5α-cholestanone, 5β-cholestanone, and cholesteryl decanoate; and mixtures thereof. In embodiments, the cholesterol derivative is a polar analogue such as cholesteryl-(4′-hydroxy)-butyl ether.

In embodiments, the lipid coating composition comprises neutral lipid species. Such lipids include, but are not limited to, cholesteryl esters, triglycerides, fatty acids, waxes, terpenes, sterol esters, vitamin A esters, glycerides, sphingomyelin, and egg sphingomyelin (ESM).

In embodiments, the lipid coating composition comprises anionic lipid species. Such lipids include, but are not limited to, POPA, phosphatidylinositol phosphates, phosphatidylserine, phosphatidyl glycerol, cardiolipin, phosphatidyl serine phosphatidic acid, danicalipin A, various fatty acids such as myristic acid, palmitic acid, stearic acid, and the like.

In embodiments, the lipid coating composition comprises phospholipids which are anionic lipid species, zwitterionic lipid species, helper lipid species, or neutral lipid species. In embodiments, phospholipids can be helper lipid species. Such lipids include, but are not limited to, 1,2-distearoyl-sn-glycero-3-phosphocholine (DSPC), 1,2-dioleoyl-sn-glycero-3-phosphoethanolamine (DOPE), or both DSPC and DOPE. Phospholipids useful in the compositions and methods described herein may be selected from the non-limiting group consisting of DSPC, DOPE, 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, lecithin, phosphatidylethanolamine, lysolecithin, lysophosphatidylethanolamine, phosphatidylserine, phosphatidylinositol, egg sphingomyelin (ESM), cephalin, cardiolipin, phosphatidic acid, cerebrosides, dicetylphosphate, dioleoylphosphatidylglycerol (DOPG), dipalmitoylphosphatidylglycerol (DPPG), palmitoyloleoyl-phosphatidylethanolamine (POPE), palmitoyloleyolphosphatidylglycerol (POPG), dioleoylphosphatidylethanolamine 4-(N-maleimidomethyl)-cyclohexane-1-carboxylate (DOPE-mal), dipalmitoyl-phosphatidylethanolamine (DPPE), dimyristoyl-phosphatidylethanolamine (DMPE), distearoyl-phosphatidylethanolamine (DSPE), monomethyl-phosphatidylethanolamine, dimethyl-phosphatidylethanolamine, dielaidoyl-phosphatidylethanolamine (DEPE), stearoyloleoyl-phosphatidylethanolamine (SOPE), lysophosphatidylcholine, dilinoleoylphosphatidylcholine, phosphatidylcholine, phosphatidic acid, phosphatidylethanolamine, phosphatidylglycerol, phosphatidylserine, and/or any derivative thereof. In yet another embodiment, the phospholipid derivatives are selected from 1,2-di-(3,7,11,15-tetramethylhexadecanoyl)-sn-glycero-3-phosphocholine, 1,2-didecanoyl-sn-glycero-3-phosphocholine, 1,2-dierucoyl-sn-glycero-3-phosphate, 1,2-dierucoyl-sn-glycero-3-phosphocholine, 1,2-dierucoyl-sn-glycero-3-phosphoethanolamine, 1,2-dilinoleoyl-sn-glycero-3-phosphocholine, 1,2-dilauroyl-sn-glycero-3-phosphate, 1,2-dilauroyl-sn-glycero-3-phosphocholine, 1,2-dilauroyl-sn-glycero-3-phosphoethanolamine, 1,2-dilauroyl-sn-glycero-3-phospho-(l′-rac-glycerol), 1,2-dimyristoyl-sn-glycero-3-phosphate, 1,2-dimyristoyl-sn-glycero-3-phosphocholine, 1,2-dimyristoyl-sn-glycero-3-phosphoethanolamine, 1,2-dimyristoyl-sn-glycero-3-phosphoglycerol, 1,2-dimyristoyl-sn-glycero-3-phosphoserine, 1,2-dioleoyl-sn-glycero-3-phosphate, 1,2-dioleoyl-sn-glycero-3-phosphocholine, 1,2-dioleoyl-sn-glycero-3-phosphoethanolamine, L-alpha-phosphatidyl-DL-glycerol, 1,2-dioleoyl-sn-glycero-3-phosphoserine, 1,2-dipalmitoyl-sn-glycero-3-phosphate, 1,2-dipalmitoyl-sn-glycero-3-phosphocholine, 1,2-dipalmitoyl-sn-glycero-3-phosphoethanolamine, 1,2-dipalmitoyl-sn-glycero-3-phosphoglycerol, 1,2-dipalmitoyl-sn-glycero-3-phosphoserine, 1,2-distearoyl-sn-glycero-3-phosphate, 1,2-distearoyl-sn-glycero-3-phosphocholine, 1,2-distearoyl-sn-glycero-3-phosphoethanolamine, 1,2-distearoyl-sn-glycero-3-phosphoglycerol, egg sphingomyelin, egg-PC, hydrogenated Egg PC, hydrogenated Soy PC, 1-myristoyl-sn-glycero-3-phosphocholine, 1-palmitoyl-sn-glycero-3-phosphocholine, 1-stearoyl-sn-glycero-3-phosphocholine, 1-myristoyl-2-palmitoyl-sn-glycero-3-phosphocholine, 1-myristoyl-2-stearoyl-sn-glycero-3-phosphocholine, 1-palmitoyl-2-myristoyl-sn-glycero-3-phosphocholine, 1-palmitoyl-2-oleoyl-sn-glycero-3-phosphocholine, 1-palmitoyl-2-oleoyl-sn-glycero-3-phosphoethanolamine, 1-palmitoyl-2-oleoyl-sn-glycero-3-phosphoglycerol, 1-palmitoyl-2-stearoyl-sn-glycero-3-phosphocholine, 1-stearoyl-2-myristoyl-sn-glycero-3-phosphocholine, 1-stearoyl-2-oleoyl-sn-glycero-3-phosphocholine, 1-stearoyl-2-palmitoyl-sn-glycero-3-phosphocholine and mixtures thereof. Other diacylphosphatidylcholine and diacylphosphatidylethanolamine phospholipids can also be used.

In embodiments, the lipid coating comprises DSPC and cholesterol. In embodiments, the lipid coating comprises DSPC. In embodiments, the lipid coating comprises DC-cholesterol.

In embodiments, the lipid coating comprises a lipid mixture of DOTAP and DOPE. In embodiments, the lipid coating comprises a lipid mixture of SM-102, DOTMA, and DSPC. In embodiments, the lipid coating comprises a lipid mixture of BAE, DOTMA, and DSPC. In embodiments, the lipid coating comprises a lipid mixture of KT-001, DOTMA, and DSPC.

In embodiments, the ratio of DOTAP to DOPE is in the range of about 1:3 to about 10:1, e.g., about 1:3, about 1:2, about 1:1, about 2:1, about 3:1, about 4:1, about 5:1, or about 10:1. In embodiments, the ratio of DOTAP:DOPE is 2.5:1 to 3.5:1, 2.7:1 to 3.3:1, 2.8:1 to 3.2:1, or 2.9:1 to 3.1:1. In embodiments, the ratio of DOTAP to DOPE is about 3:1. In embodiments, the ratio of DOTAP to DOPE is 3:1.

In embodiments, the ratio of SM-102 to DOTMA to DSPC may be between about 40-80:1-20:2-40. In embodiments, the ratio of SM-102:DOTMA:DSPC is 5:1:2 to 10:1:2, 5:2:4 to 10:2.5:6, 6:1:3 to 8:1:3, or 6:1:1 to 8:1:4. In embodiments, the ratio of SM-102:DOTMA:DSPC is 7:1:2.

In embodiments, the ratio of BAE to DOTMA to DSPC may be between about 60-120:1-20:2-40. In embodiments, the ratio of BAE:DOTMA:DSPC is 5:1:2 to 15:1:2, 5:1:1 to 15:1:4, or 8:1:2 to 12:1:4. In embodiments, the ratio of BAE:DOTMA:DSPC is 10.4:1:2.

In embodiments, the ratio of KT-001 to DOTMA to DSPC may be between about 50-90:about 1-20:about 2-40. In embodiments, the ratio of KT-001:DOTMA:DSPC is 5:1:2 to 15:1:2, 5:1:1 to 15:1:4, or 8:1:2 to 12:1:4. In embodiments, the ratio of KT-001:DOTMA:DSPC is 7.9:1:2

In embodiments, the selection of ionizable cationic lipid species, a cationic lipid species, an anionic lipid species, a neutral lipid species, and/or a helper lipid species which comprise the lipid coating, as well as the relative molar ratio of such lipid species to each other, is based upon the characteristics of the selected lipid(s) species, the nature of the intended target cells, pH and the like.

In embodiments herein, the terms “lipids” and “lipid species” are used interchangeably. Any desired lipid species may be mixed at any ratios suitable for encapsulating nanoparticles. In embodiments, a suitable lipid solution contains a mixture of desired lipid species including ionizable cationic lipid species, a cationic lipid species, an anionic lipid species, a neutral lipid species, and/or a helper lipid species. In embodiments, a suitable lipid solution contains a mixture of desired lipid species including one or more cationic lipids, one or more helper lipids, one or more anionic lipids, one or more ionizable cationic lipids, and one or more neutral lipids. In embodiments, a suitable lipid solution contains a mixture of desired lipid species including one or more neutral lipids and one or more helper lipids. In embodiments, a suitable lipid solution contains one or more helper lipids.

In embodiments, the nanoparticles containing the desired payload, such as imaging agents, small molecules or other therapeutic agents, are encapsulated in a liposome or a micelle. In embodiments, the surface of the liposome or the micelle can be modified. In embodiments, the surface of the liposome or the micelle can be modified with a targeting ligand.

In embodiments, the surface of the liposome or the micelle can be modified with PEG. In embodiments, the surface of the liposome can be modified with PEG. In embodiments, the surface of the micelle can be modified with PEG. In embodiments, the surface of the liposome or the micelle can be modified with PEG whose distal end is connected to a targeting ligand. In embodiments, the surface of the liposome can be modified with PEG whose distal end is connected to a targeting ligand. In embodiments, the surface of the micelle can be modified with PEG whose distal end is connected to a targeting ligand. In embodiments, the targeting ligand is a small molecule, a protein, a peptide, an antibody, or a carbohydrate.

In embodiments, the surface of the liposome is modified with PEG linked to a small molecule. In embodiments, the surface of the liposome is modified with PEG linked to a protein. In embodiments, the surface of the liposome is modified with PEG linked to an antibody. In embodiments, the surface of the liposome is modified with PEG linked to a peptide. In embodiments, the surface of the liposome is modified with PEG is linked to a carbohydrate.

In embodiments, the surface of the micelle is modified with PEG linked to a small molecule. In embodiments, the surface of the micelle is modified with PEG linked to a protein. In embodiments, the surface of the micelle is modified with PEG linked to an antibody. In embodiments, the surface of the micelle is modified with PEG linked to a peptide. In embodiments, the surface of the micelle is modified with PEG is linked to a carbohydrate.

In embodiments, the surface of the liposome or the micelle can be modified with a protein. In embodiments, the surface of the liposome or the micelle can be modified with an antibody. In embodiments, the surface of the liposome or the micelle can be modified with a peptide. In embodiments, the surface of the liposome or the micelle can be modified with a carbohydrate. In embodiments, the surface of the liposome or the micelle can be modified with a small molecule.

In embodiments, the liposome contains an imaging agent, and its surface is modified with a targeting ligand. In embodiments, the liposome contains an imaging agent, and its surface is modified with a peptide. In embodiments, the liposome contains an imaging agent, and its surface is modified with a protein. In embodiments, the liposome contains an imaging agent, and its surface is modified with a small molecule. In embodiments, the liposome contains an imaging agent, and its surface is modified with an antibody. In embodiments, the liposome contains an imaging agent, and its surface is modified with a carbohydrate.

In embodiments, the micelle contains an imaging agent, and its surface is modified with a targeting ligand. In embodiments, the micelle contains an imaging agent, and its surface is modified with a peptide. In embodiments, the micelle contains an imaging agent, and its surface is modified with a protein. In embodiments, the micelle contains an imaging agent, and its surface is modified with a small molecule. In embodiments, the micelle contains an imaging agent, and its surface is modified with an antibody. In embodiments, the micelle contains an imaging agent, and its surface is modified with a carbohydrate.

In embodiments, the surface of the liposome or the micelle is modified with a functionalized imaging agent. In embodiments, the surface of the liposome is modified with a functionalized imaging agent. In embodiments, the surface of the micelle is modified with a functionalized imaging agent. In embodiments, the imaging agent is functionalized with a targeting ligand. In embodiments, the imaging agent is functionalized with an antibody. In embodiments, the imaging agent is functionalized with a peptide. In embodiments, the imaging agent is functionalized with a protein. In embodiments, the imaging agent is functionalized with a carbohydrate. In embodiments, the imaging agent is functionalized with a small molecule.

### Nanoparticles

In embodiments, provided herein is a nanoparticle containing entrapped payload molecules. In embodiments, the nanoparticle comprises lipids. In embodiments, the nanoparticle is a lipid nanoparticle (LNP). In embodiments, the nanoparticle is a solid lipid nanoparticle. In embodiments, the nanoparticle is an emulsion nanoparticle.

In embodiments, the nanoparticles are lipid-based nanoparticles. Non-limiting examples of lipid-based nanoparticles contemplated herein include lipid nanoparticles (LNPs), solid lipid nanoparticles, SLNs), emulsion nanoparticles (nanoemulsions), micelles, and liposomes. In embodiments, the nanoparticles are inorganic nanoparticles. Non-limiting examples of inorganic nanoparticles contemplated herein include iron oxide nanoparticles, gold nanoparticles, silica nanoparticles, mesoporous silica nanoparticles, and quantum dots. In embodiments, the nanoparticles are polymeric nanoparticles. Non-limiting examples of polymeric nanoparticles contemplated herein include dendrimers, polymer micelles, poly(lactic-co-glycolic acid) (PLGAs), methoxypoly(ethylene glycol)-poly(glycerol adipate) (MPEG-PGA), poly(ethylene glycol)-poly(ω-pentadecalactone-co-N-methyldiethyleneamine sebacate-co-2,2′-thiodiethylene sebacate) (PEG-PMT), and polyethyleneimine-poly(lactic-co-glycolic acid) (PEI-PLGA).

In embodiments, the nanoparticles are lipid nanoparticles (LNPs), where a lipid shell surrounds an internal core composed of reverse micelles that encapsulate payload molecules, as shown in FIG. 2. In embodiments, the nanoparticles are solid lipid nanoparticles (SLNs), where a surfactant shell surrounds an internal core composed of solid lipids. In embodiments, the nanoparticles are emulsion nanoparticles, where a surfactant shell surrounds an internal core composed of liquid lipids.

In embodiments, the lipid-based nanoparticle comprises an ionizable cationic lipid, a helper lipid, and optionally a cholesterol and/or a PEG. In embodiments, the lipid-based nanoparticle comprises an ionizable cationic lipid and a helper lipid. In embodiments, the lipid-based nanoparticle comprises an ionizable cationic lipid and a sterol. In embodiments, the lipid-based nanoparticle comprises an ionizable cationic lipid and a cholesterol. In embodiments, the lipid-based nanoparticle comprises an ionizable cationic lipid, a cholesterol, and a PEG. In embodiments, the lipid-based nanoparticle comprises an ionizable cationic lipid.

In embodiments, the lipid nanoparticle (LNP) comprises an ionizable cationic lipid, a helper lipid, and optionally a cholesterol and/or a PEG. In embodiments, the LNP comprises an ionizable cationic lipid and a helper lipid. In embodiments, the LNP comprises an ionizable cationic lipid and a sterol. In embodiments, the LNP comprises an ionizable cationic lipid and a cholesterol. In embodiments, the LNP comprises an ionizable cationic lipid, a cholesterol, and a PEG. In embodiments, the LNP comprises an ionizable cationic lipid. In embodiments, the LNP comprises a neutral lipid and optionally a helper lipid. In embodiments, the LNP comprises a neutral lipid.

In embodiments, the LNP comprises KT-001, DSPC, cholesterol, and DMG-PEG2000. In embodiments, the LNP comprises KT-001. In embodiments, the LNP comprises KT-001 and a helper lipid. In embodiments, the LNP comprises KT-001 and cholesterol. In embodiments, the LNP comprises DSPC and a helper lipid. In embodiments, the LNP comprises DSPC and cholesterol. In embodiments, the LNP comprises DSPC. In embodiments, percentage of PEG in the LNP is from 0 to about 5 percent. In embodiments, the percentage of PEG in the LNP is from 0 to about 2 percent. In embodiments, percentage of PEG in the LNP is 0. In embodiments, the percentage of PEG in the LNP is about 0.5%. In embodiments, the percentage of PEG in the LNP is about 1.0%. In embodiments, the percentage of PEG in the LNP is about 1.5%. In embodiments, the percentage of PEG in the LNP is about 2.5%. In embodiments, the percentage of PEG in the LNP is about 2.5%. In embodiments, the percentage of PEG in the LNP is about 3.0%. In embodiments, percentage of PEG in the LNP is about 4.0%. In embodiments, the percentage of PEG in the LNP is about 5.0%.

In embodiments, the ratio of KT-001:DSPC:cholesterol:DMG-PEG2000 is about 49.9:10:38.4:0.17, about 40:5:53:0.2, about 60:10:28:0.2, or about 45:8:44:1. In embodiments, the ratio of KT-001:DSPC:cholesterol:DMG-PEG2000 is 49.9:10:38.4:0.17.

In embodiments, the ratio of KT-001 to DSPC to cholesterol to DMG-PEG2000 may be between about 40-60:5-15:31-45:0.05-2.

In embodiments, the emulsion nanoparticle comprises an ionizable cationic lipid, a helper lipid, and optionally a cholesterol and/or a PEG. In embodiments, the emulsion nanoparticle comprises an ionizable cationic lipid and a helper lipid. In embodiments, the emulsion nanoparticle comprises an ionizable cationic lipid and a sterol. In embodiments, the emulsion nanoparticle comprises an ionizable cationic lipid and a cholesterol. In embodiments, the emulsion nanoparticle comprises an ionizable cationic lipid, a cholesterol, and a PEG. In embodiments, the emulsion nanoparticle comprises an ionizable cationic lipid and a surfactant.

In embodiments, the surfactant is TPGS1000. In embodiments, the emulsion nanoparticle comprises SM-102 and TPGS1000.

In embodiments, the ratio of SM-102:TPGS1000 is about 62.5:37.5, about 55:45, about 70:30, about 80:20, or about 50:50. In embodiments, the ratio of SM-102:TPGS1000 is 62.5:37.5. In embodiments, the ratio of SM-102 to TPGS1000 may be between 50-70:50:30.

In embodiments, the lipid-based nanoparticle encapsulates a plurality of payload molecules. In embodiments, the payload molecules are hydrophilic. In embodiments, the payload molecules are hydrophobic. In embodiments, the payload molecules are imaging agents, small molecules, or other therapeutic agents. In embodiments, the payload molecules are imaging agents, small molecules, nucleic acids, peptides, or proteins. In embodiments, the payload molecules are imaging agents or detectable moieties.

In embodiments, the payload molecules are imaging agents. In embodiments, the payload molecules are detectable moieties. Non-limiting examples of imaging agents and detectable moieties contemplated herein include green fluorescent protein (GFP), enhanced cyan fluorescent protein (ECFP), DsRed fluorescent protein (DsRed2FP), enhanced green fluorescent protein (EGFP), enhanced yellow fluorescent protein (EYFP), radioactive isotopes (e.g., Copper-64, fluorine-18 (FDG-18), Technetium-99, zirconium (Zr-95, Zr-88, Zr-89)), and the like. In embodiments, the payload molecules are green fluorescent protein (GFP).

In embodiments, the payload molecules are small molecules. Non-limiting examples of small molecules contemplated herein include small molecule drugs e.g., anti-cancer drugs, anti-bacterial drugs, anti-viral drugs and the like. Non-limiting examples of anti-cancer drugs contemplated herein include doxorubicin, daunorubicin, edelfosine, gemcitabine, vincristine, oxiplatin, irinotecan, carboplatin, paclitaxel, docetaxel, cisplatin, etoposide, methotrexate, 5-fluorouracil, and the like. Non-limiting examples of anti-bacterial drugs contemplated herein include penicillins, tetracyclines, lincomycins, glycopeptides, aminoglycosides, cephalosporins, quinolones, sulfonamides, carbapenems, and the like.

In embodiments, the payload molecules are therapeutic agents. Non-limiting examples of therapeutic agents contemplated herein include proteins, peptides, aptamers, enzymes, prodrugs, nucleic acids e.g., DNA, RNA, mRNA, siRNA, shRNA, and the like.

The term “payload” refers to any chemical entity, pharmaceutical, imaging agent, drug (such drug can be, but not limited to, a small molecule, an inorganic solid, a polymer, or a biopolymer), small molecule, nucleic acid (e.g., DNA, RNA, mRNA, etc.), protein, peptide and the like that is entrapped within the lipid nanoparticle described in the present disclosure. In various embodiments, the payload is comprised of one or more nucleotides. For example, in various embodiments, the payload is an oligonucleotide.

In various embodiments, the payload is an oligonucleotide. In various embodiments, the oligonucleotide is an antisense molecule. In various embodiments, the oligonucleotide is a siRNA. In various embodiments, the oligonucleotide is a shRNA. In various embodiments, the oligonucleotide is a DNA. In various embodiments, the oligonucleotide is an RNA. In various embodiments, the oligonucleotide is an mRNA. The oligonucleotide may be of a varied length. In various embodiments, the oligonucleotide is about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, or about 40 nucleotides in length. In various embodiments, the oligonucleotide is between about 2 and about 40 nucleotides in length. In various embodiments, the oligonucleotide is between about 4 and about 35 nucleotides in length. In various embodiments, the oligonucleotide is about 10 and about 30 nucleotides in length. In various embodiments, the oligonucleotide is between about 12 and about 17 nucleotides in length.

In various embodiments, the payload is an mRNA. In various embodiments, that mRNA is about 500-3000 nucleotides in length. In various embodiments, the mRNA is 500 nucleotides, 1000 nucleotides, 1500 nucleotides, 2000 nucleotides, 2500 nucleotides, 3000 nucleotides in length. In various embodiments, the mRNA encodes an antigenic peptide.

In various embodiments, the payload is a polypeptide. In various embodiments, the polypeptide is between about 1,000 and 10,000 Da. In various embodiments, the polypeptide is about 500 Da, about 600 Da, about 700 Da, about 800 Da, about 900 Da, about 1,000 Da, about 1,500 Da, about 2,000 Da, about 2,500 Da, about 3,000 Da, about 3,500 Da, about 4,000 Da, about 4,500 Da, about 5,000 Da, about 5,500 Da, about 6,000 Da, about 6,500 Da, about 7,000 Da, about 7,500 Da, about 8,000 Da, about 8,500 Da, about 9,000 Da, about 9,500 Da, about 10,000 Da, about 15,000 Da or about 20,000 Da.

In various embodiments, the payload is a small molecule. In various embodiments, the small molecule is between about 100 Da and 1000 Da. In various embodiments, the small molecule is about 50 Da, about 60 Da, about 70 Da, about 80 Da, about 90 Da, about 100 Da, about 150 Da, about 200 Da, about 250 Da, about 300 Da, about 350 Da, about 400 Da, about 450 Da, about 500 Da, about 550 Da, about 600 Da, about 650 Da, about 700 Da, about 750 Da, about 800 Da, about 850 Da, about 900 Da, about 950 Da, about 1,000 Da, about 1,500 Da or about 2,000 Da.

In embodiments, when a liposome contains the nanoparticle, a hydrophilic drug is contemplated in the aqueous core of the liposome in addition to the nanoparticle. In embodiments, when a liposome contains the nanoparticle, a hydrophobic drug is contemplated in the lipid bilayer of the liposome and the nanoparticle in the aqueous core of the liposome.

In embodiments, when a lipid micelle contains the nanoparticle, a hydrophobic drug is contemplated in the hydrophobic core of the micelle in addition to the nanoparticle.

### Pharmaceutical Compositions

In an aspect, provided herein are pharmaceutical compositions comprising the lipid-coated nanoparticle as described herein, including in embodiments, and a pharmaceutically acceptable excipient.

In embodiments, the lipid-coated nanoparticle described herein, may be formulated as a pharmaceutical composition. The pharmaceutical composition may include one or more lipid-coated nanoparticle compositions. In embodiments, pharmaceutical composition may include one or more lipid-coated nanoparticle compositions including one or more different payloads. Pharmaceutical compositions may further include one or more pharmaceutically acceptable excipients or accessory ingredients such as those described herein. General guidelines for the formulation and manufacture of pharmaceutical compositions and agents are available, for example, in Remington's The Science and Practice of Pharmacy, 21st Edition, A. R. Gennaro; Lippincott, Williams & Wilkins, Baltimore, Md., 2006.

Relative amounts of the one or more lipid-coated nanoparticle compositions, the one or more pharmaceutically acceptable excipients, and/or any additional ingredients in a pharmaceutical composition in accordance with the present disclosure will vary, depending upon the identity, size, and/or condition of the subject treated and further depending upon the route by which the composition is to be administered. By way of example, a pharmaceutical composition may comprise between 0.1% and 100% (wt/wt) of one or more lipid-coated nanoparticle compositions.

Non-limiting examples of pharmaceutically acceptable excipients include water for injection (WFI), NaCl, PBS, normal saline solutions, lactated Ringer's, normal sucrose, normal glucose, binders, fillers, disintegrants, lubricants, coatings, sweeteners, flavors, salt solutions (such as Ringer's solution), alcohols, fixed oils, polyethylene glycols, glycerine, propylene glycol, gelatins, carbohydrates such as lactose, amylose or starch, fatty acid esters, hydroxymethycellulose, polyvinyl pyrrolidine, and colors, and the like.

The pharmaceutically acceptable excipient is usually added following lipid-coated nanoparticle formation. Thus, after the lipid-coated nanoparticle is formed, it can be diluted into pharmaceutically acceptable excipients such as normal buffered saline. Pharmaceutical compositions typically include a conventional pharmaceutical excipient and may additionally include other medicinal agents, carriers, adjuvants, additives and the like.

Pharmaceutical compositions may be prepared in a variety of forms suitable for a variety of routes and methods of administration. For example, pharmaceutical compositions of the invention may be prepared in liquid dosage forms (e.g., emulsions, microemulsions, nanoemulsions, solutions, suspensions, syrups, and elixirs), injectable forms, solid dosage forms (e.g., capsules, tablets, pills, powders, and granules), dosage forms for topical and/or transdermal administration (e.g., ointments, pastes, creams, lotions, gels, powders, solutions, sprays, inhalants, and patches), suspensions, powders, and other forms. Liquid dosage forms for oral and parenteral administration include, but are not limited to, pharmaceutically acceptable emulsions, microemulsions, nanoemulsions, solutions, suspensions, syrups, and/or elixirs. In addition to active ingredients, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and suspending agents, sweetening, flavoring, and/or perfuming agents. In certain embodiments for parenteral administration, compositions are mixed with solubilizing agents such as Cremophor®, alcohols, oils, modified oils, glycols, polysorbates, cyclodextrins, polymers, and/or combinations thereof.

Pharmaceutical formulations containing the lipid-coated nanoparticles may take the form of liquid, solid, semi-solid or lyophilized powder forms, such as, for example, solutions, suspensions, emulsions, sustained-release formulations, tablets, capsules, powders, suppositories, creams, ointments, lotions, aerosols, patches or the like, e.g., in unit dosage forms suitable for simple administration of precise dosages.

For in vivo administration, administration can be in any manner known in the art, e.g., by injection, oral administration, inhalation (e.g., intranasal or intratracheal), transdermal application (topical), transmucosal, or rectal administration.

In embodiments, the pharmaceutical compositions can be administered parenterally, e.g., intraarticularly, intravenously, intradermally, intrathecally, intraperitoneally, subcutaneously, or intramuscularly. In embodiments, the pharmaceutical compositions are administered intravenously or intraperitoneally by a bolus injection. In embodiments, parenteral preparation is enclosed in ampules, disposable syringes or multiple dose vials made of glass or plastic.

An injectable composition for parenteral administration (e.g., intravenous, intramuscular or intrathecal) will typically contain the compound in a suitable i.v. solution, such as sterile physiological salt solution. The composition may also be formulated as a suspension in an aqueous emulsion.

In an aspect, provided herein is a method for preventing or treating a disease in a mammal in need thereof by administering to the mammal a therapeutically effective amount of a lipid-coated lipid nanoparticles (LC-LNPs) as described herein including in embodiments. In embodiments, provided herein is a method for preventing a disease in a mammal by administering to the mammal a therapeutically effective amount of lipid-coated lipid nanoparticles (LC-LNPs) as described herein including in embodiments. In embodiments, provided herein is a method for treating a disease in a mammal, in need thereof, by administering to the mammal a therapeutically effective amount of a lipid-coated lipid nanoparticles (LC-LNPs) as described herein including in embodiments.

In embodiments the mammal is a dog, a cat, or a human. In embodiments, the mammal is a dog. In embodiments, the mammal is a cat. In embodiments, the mammal is a human.

### Preparation of Lipid-Coated Nanoparticles

In another aspect, provided herein is a method for manufacturing lipid-coated lipid nanoparticles (LNPs) comprising:
(a) dissolving at least one payload molecule into a first solution or a second solution, wherein the first solution comprises an aqueous phase and the second solution comprises an organic phase and a plurality of molecules capable of self-assembly, and wherein the first and second solutions are miscible;
(b) mixing the first solution and the second solution using microfluidics to obtain lipid nanoparticles encapsulating the at least one payload molecule under conditions suitable for LNP formation, thereby forming LNPs;
(c) purifying said LNPs;
(d) adjusting LNP concentration in an aqueous phase; and
(e) mixing said LNPs and a third solution using microfluidics to obtain lipid-coated LNPs, wherein the third solution comprises an organic phase and a plurality of molecules capable of self-assembly, and the third solution contains the same or different molecules as the second solution.

In another aspect, provided herein is a method for manufacturing lipid-coated lipid nanoparticles (LNPs) comprising mixing LNPs with a third solution using microfluidics to obtain lipid-coated LNPs, wherein:
the LNPs were formed by mixing a first solution and a second solution using microfluidics under conditions suitable for LNP formation to obtain lipid nanoparticles encapsulating at least one payload molecule;
the first solution comprises an aqueous phase and the second solution comprises an organic phase and a plurality of molecules capable of self-assembly;
the first and second solutions are miscible;
the at least one payload molecule was dissolved in the first solution or the second solution;
the third solution comprises an organic phase and a plurality of molecules capable of self-assembly, and the third solution contains the same or different molecules as the second solution.

In yet another aspect, provided herein is a method for manufacturing lipid-coated lipid nanoparticles (LNPs) comprising:
(a) preparing a first solution comprising an aqueous phase;
(b) preparing a second solution comprising an organic phase and a plurality of molecules capable of self-assembly, and wherein the first and second solutions are miscible;
(c) dissolving at least one payload molecule into the first or second solution;
(d) mixing said first and second solutions using microfluidics to obtain lipid nanoparticles encapsulating said payload under conditions suitable for LNP formation;
(e) purifying said LNPs;
(f) adjusting LNP concentration in aqueous phase;
(g) preparing a third solution comprising an organic phase and a plurality of molecules capable of self-assembly, wherein the third solution contains the same or different molecules as the second solution; and
(h) mixing said LNPs and third solution using microfluidics to obtain lipid-coated LNPs.

In embodiments, the lipid-coated lipid nanoparticles (LNPs) are purified after the manufacturing process.

In embodiments, the method for manufacturing lipid-coated lipid nanoparticles can be applied for manufacturing other lipid-coated nanoparticles such as, for example, lipid-coated solid lipid nanoparticles or lipid-coated nanoemulsions.

The process for preparing solid lipid nanoparticles and nanoemulsions has been previously described, for example, by Calva-Estrada et al., 2017, J. Disper. Sci. Technol. 39:181-189; Galvão et al., 2018, 11:355-367 doi: 10.1007/s11947-017-2016-y; Shegokar et al., 2011, Int. J. Pharm. 416:461-470; and Van-an et al., 2020, Molecules 25:4781-4812.

In embodiments, the method for manufacturing lipid-coated lipid-based nanoparticles comprises a first process of preparing lipid-based nanoparticles, and a second process of coating said nanoparticles with lipids. In embodiments, the method for manufacturing lipid-coated lipid nanoparticles (LC-LNPs) comprises a first process of preparing lipid nanoparticles (LNPs), and a second process of coating said nanoparticles with lipids. In embodiments, the method for manufacturing lipid-coated nanoemulsion comprises a first process of preparing nanoemulsion, and a second process of coating said nanoemulsion with lipids. In embodiments, the method for manufacturing lipid-coated solid lipid nanoparticles (LC-SLNs) comprises a first process of preparing solid lipid nanoparticles (SLNs), and a second process of coating said nanoparticles with lipids.

In embodiments, the first process involves a first step where an aqueous solution is prepared. In embodiments, the aqueous solution is an aqueous buffer. In embodiments, the aqueous buffer is an acetate buffer, citric buffer, Tyrode's buffer, TBT buffer, TBS buffer, or TBS sucrose buffer. In embodiments, the aqueous buffer is an acetate buffer. In embodiments, the aqueous buffer is sodium acetate buffer adjusted to pH 5.

In embodiments, the first process involves a second step of preparing a second solution comprising an organic phase and a plurality of molecules capable of self-assembly, and wherein the first and second solutions are miscible. In embodiments, the first process involves a second step where lipids are dissolved in a water-miscible organic solvent. In embodiments, the water miscible organic solvent is ethanol, methanol, acetone, acetonitrile, ethylamine, glycerol, or dioxane. In embodiments, the water miscible organic solvent is ethanol or methanol. In embodiments, the water miscible organic solvent is ethanol. In embodiments, the water miscible organic solvent is methanol.

In embodiments, the payload molecules are dissolved in the first or second solution. In embodiments, at least one payload molecule is dissolved in the first or second solution. In embodiments, at least one payload molecule is dissolved in the first solution. In embodiments, at least one payload molecule is dissolved in the second solution. In embodiments, a plurality of payload molecules is dissolved in the first solution. In embodiments, a plurality of payload molecules is dissolved in the second solution.

In embodiments, the payload molecules are hydrophilic. In embodiments, the payload molecules are hydrophobic. In embodiments, the payload molecules are imaging agents, small molecules, or other therapeutic agents. In embodiments, the payload molecules are imaging agents, small molecules, nucleic acids, peptides, or proteins. In embodiments, the payload molecules are imaging agents or detectable moieties.

In embodiments, the payload molecules are imaging agents. In embodiments, the payload molecules are detectable moieties. Non-limiting examples of imaging agents and detectable moieties contemplated herein include green fluorescent protein (GFP), enhanced cyan fluorescent protein (ECFP), DsRed fluorescent protein (DsRed2FP), enhanced green fluorescent protein (EGFP), enhanced yellow fluorescent protein (EYFP), radioactive isotopes (e.g., Copper-64, fluorine-18 (FDG-18), Technetium-99, zirconium (Zr-95, Zr-88, Zr-89)), and the like. In embodiments, the payload molecules are green fluorescent protein (GFP).

In embodiments, the payload molecules are small molecules. Non-limiting examples of small molecules contemplated herein include small molecule drugs e.g., anti-cancer drugs, anti-bacterial drugs, anti-viral drugs and the like. Non-limiting examples of anti-cancer drugs contemplated herein include doxorubicin, daunorubicin, edelfosine, gemcitabine, vincristine, oxiplatin, irinotecan, carboplatin, paclitaxel, docetaxel, cisplatin, etoposide, methotrexate, 5-fluorouracil, and the like. Non-limiting examples of anti-bacterial drugs contemplated herein include penicillins, tetracyclines, lincomycins, glycopeptides, aminoglycosides, cephalosporins, quinolones, sulfonamides, carbapenems, and the like.

In embodiments, the payload molecules are therapeutic agents. Non-limiting examples of therapeutic agents contemplated herein include proteins, peptides, aptamers, enzymes, prodrugs, and nucleic acids e.g., DNA, RNA, mRNA, siRNA, shRNA, and the like.

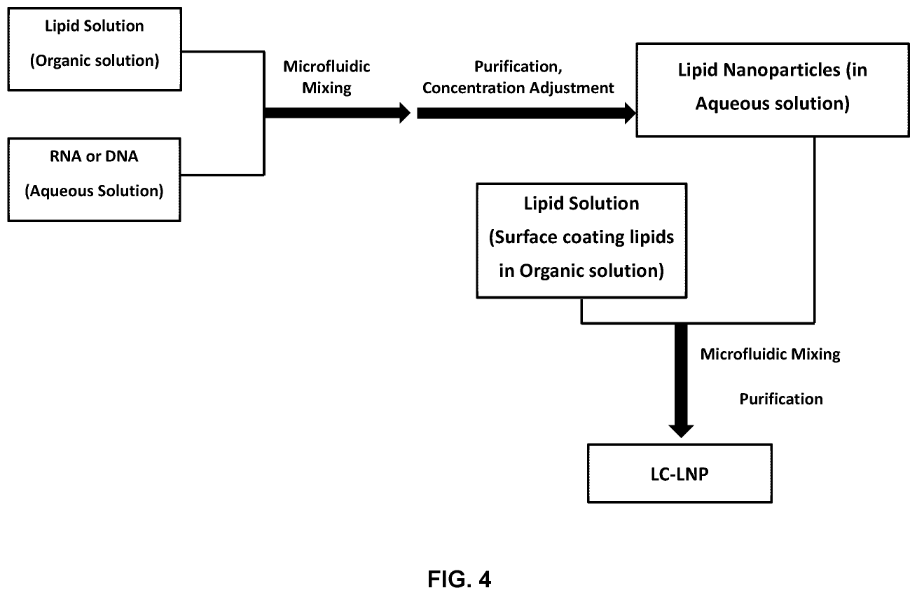

In embodiments, the first and second solutions are mixed using microfluidics, as shown in FIG. 4, to obtain lipid nanoparticles encapsulating the payload under conditions suitable for LNP formation. In embodiments, the LNPs are purified. In embodiments, the purification of LNPs is done via dialysis. In embodiments, the concentration of LNPs is adjusted before coating them with lipid. In embodiments, the concentration of LNPs is adjusted with TBS sucrose buffer (pH 7.3-7.4).

In embodiments, the first process involves a first step of preparing an aqueous solution. In embodiments, the aqueous solution is an aqueous buffer. In embodiments, the aqueous buffer is an acetate buffer, citric buffer, Tyrode's buffer, TBT buffer, TBS buffer, or TBS sucrose buffer. In embodiments, the aqueous buffer is an acetate buffer.

In embodiments, the first process involves a second step of preparing a second solution comprising an organic phase and a plurality of molecules capable of self-assembly, and wherein the first and second solutions are miscible. In embodiments, the molecules capable of self-assembly are lipids, proteins, or polymers. In embodiments, the molecules capable of self-assembly are lipids. In embodiments, the molecules capable of self-assembly are proteins. In embodiments, the molecules capable of self-assembly are polymers.

In embodiments, the first process involves a second step where lipids are dissolved in a water-miscible organic solvent. In embodiments, the water miscible organic solvent is ethanol, methanol, acetone, acetonitrile, ethylamine, glycerol, or dioxane. In embodiments, the water miscible organic solvent is ethanol or methanol. In embodiments, the water miscible organic solvent is ethanol. In embodiments, the water miscible organic solvent is methanol.

In embodiments, the payload molecules are dissolved in the first or second solution. In embodiments, at least one payload molecule is dissolved in the first or second solution. In embodiments, at least one payload molecule is dissolved in the first solution. In embodiments, at least one payload molecule is dissolved in the second solution. In embodiments, a plurality of payload molecules is dissolved in the first solution. In embodiments, a plurality of payload molecules is dissolved in the second solution. In embodiments, the payload molecules are absent.

In embodiments, the first process involves a first step of preparing an aqueous solution comprising a plurality of payload molecules and a second step of preparing a second solution comprising an organic phase and a plurality of molecules capable of self-assembly, and wherein the first and second solutions are miscible. In embodiments, the first process involves a first step of preparing an aqueous solution and a second step of preparing a second solution comprising an organic phase a plurality of molecules capable of self-assembly and a plurality of payload molecules, and wherein the first and second solutions are miscible.

In embodiments, the first process involves a second step where lipids and payload molecules are dissolved in the second solution. In embodiments, the first process involves a second step where lipids and payload molecules are dissolved in a water-miscible organic solvent. In embodiments, the lipid and payload solution prepared in the second step is added to the first solution (an aqueous solution as described above) while being mixed by a high shear homogenizer. In embodiments, said solution is then added to a microfluidizer to create a nanoemulsion. In embodiments, the concentration of the nanoemulsion is adjusted before coating with lipid. In embodiments, the concentration of nanoemulsion is adjusted with Tyrode's buffer.

In embodiments, the second process involves preparing a third solution comprising an organic phase and a plurality of molecules capable of self-assembly, wherein the third solution contains the same or different molecules as the second solution. In embodiments, the second process involves preparing a third solution where lipids are dissolved in a water-miscible organic solvent. In embodiments, the lipids in the third solution are the same or different than the lipids in the second solution. In embodiments, the lipids in the third solution are the same as the lipids in the second solution. In embodiments, the lipids in the third solution are different than the lipids in the second solution. In embodiments, the third solution is miscible with the aqueous solution containing lipid-based nanoparticles. In embodiments, the third solution is miscible with the aqueous solution containing LNPs. In embodiments, the third solution is miscible with the aqueous solution containing nanoemulsion. In embodiments, the third solution is miscible with the aqueous solution containing SLNs.

In embodiments, the lipid-based nanoparticles and the third solution are mixed using microfluidics, as shown in FIG. 4, to obtain lipid-coated lipid-based nanoparticles. In embodiments, the lipid nanoparticles (LNPs) and the third solution are mixed using microfluidics, to obtain lipid-coated lipid nanoparticles (LC-LNPs). In embodiments, the nanoemulsion and the third solution are mixed using microfluidics, to obtain lipid-coated nanoemulsion. In embodiments, the solid lipid nanoparticles (SLNs) and the third solution are mixed using microfluidics, to obtain lipid-coated solid lipid nanoparticles.

In embodiments, once lipid-coated lipid-based nanoparticles are formed, they are incubated to allow for stabilization. In embodiments, once lipid-coated lipid nanoparticles (LC-LNPs) are formed, they are incubated to allow for stabilization. In embodiments, once lipid-coated solid lipid nanoparticles are formed, they are incubated to allow for stabilization. In embodiments, once lipid-coated nanoemulsions are formed, they are incubated to allow for stabilization. In embodiments, the incubation is for about 1, 2, 5, 10, 15, 20, 30, 40, 60, 90, 120, 180, or 240 minutes. In embodiments, the incubation is for about 1 minute. In embodiments, the incubation is for about 2 minutes. In embodiments, the incubation is for about 10 minutes. In embodiments, the incubation is for about 20 minutes. In embodiments, the incubation is for about 30 minutes. In embodiments, the incubation is for about 60 minutes. In embodiments, the incubation is for about 90 minutes. In embodiments, the incubation is for about 120 minutes. In embodiments, the incubation is for about 180 minutes. In embodiments, the incubation is for about 240 minutes.

In embodiments, the lipid-coated lipid-based nanoparticles are purified. In embodiments, the LC-LNPs are purified. In embodiments, the LC-SLNs are purified. In embodiments, the LC-nanoemulsions are purified. In embodiments, the purification of LC-LNPs is done via dialysis. In embodiments, the purification of LC-SLNs is done via dialysis. In embodiments, the purification of LC-nanoemulsions is done via dialysis.

In embodiments, the self-assembling molecules include at least a lipid component comprised of at least one species of lipid molecule. In embodiments, the at least one species of lipid molecule is selected from an ionizable cationic lipid species, a cationic lipid species, an anionic lipid species, a neutral lipid species, and a helper lipid species. In embodiments, the at least one species of lipid molecule is an ionizable cationic lipid species. In embodiments, the at least one species of lipid molecule is a cationic lipid species. In embodiments, the at least one species of lipid molecule is an anionic lipid species. In embodiments, the at least one species of lipid molecule is a neutral lipid species. In embodiments, the at least one species of lipid molecule is a helper lipid species.

In embodiments, the second solution includes two, three, or four species of lipid molecules, wherein the species of lipid molecules are selected from an ionizable cationic lipid species, a cationic lipid species, an anionic lipid species, a neutral lipid species, a helper lipid species, or any combination thereof.

In embodiments, the second solution comprises an ionizable cationic lipid, a helper lipid, and optionally a cholesterol and/or a PEG. In embodiments, the second solution comprises an ionizable cationic lipid and a helper lipid. In embodiments, the percentage of ionizable cationic lipid is about 25% to about 75%. In embodiments, the second solution comprises an ionizable cationic lipid and a sterol. In embodiments, the second solution comprises an ionizable cationic lipid and a cholesterol. In embodiments, the second solution comprises an ionizable cationic lipid, a cholesterol, and a PEG. In embodiments, the second solution comprises an ionizable cationic lipid. In embodiments, the second solution comprises a neutral lipid and optionally a helper lipid. In embodiments, the second solution comprises a neutral lipid. In embodiments, the second solution comprises an ionizable cationic lipid and a surfactant.

In embodiments, the second solution comprises KT-001, DSPC, cholesterol, and DMG-PEG2000. In embodiments, the second solution comprises KT-001. In embodiments, the second solution comprises KT-001 and a helper lipid. In embodiments, the second solution comprises KT-001 and cholesterol. In embodiments, the second solution comprises KT-001, DSPC and cholesterol. In embodiments, the second solution comprises KT-001, DSPC and DC-cholesterol. In embodiments, the second solution comprises KT-001, DOPC and cholesterol.

In embodiments, the second solution comprises DSPC and a helper lipid. In embodiments, the second solution comprises DOPC and a helper lipid. In embodiments, the second solution comprises DSPC and cholesterol. In embodiments, the second solution comprises DSPC. In embodiments, the second solution comprises DC-cholesterol and DSPC. In embodiments, the second solution comprises DOPC and cholesterol.

In embodiments, the second solution comprises KT-001, DSPC, cholesterol, and DMG-PEG2000 at a ratio of about 49.9:10:38.4:0.17, about 40:5:53:0.2, about 60:10:28:0.2, or about 45:8:44:1. In embodiments, the second solution comprises KT-001, DSPC, cholesterol, and DMG-PEG2000 at a ratio of 49.9:10:38.4:0.17.

In embodiments, the second solution comprises KT-001, DSPC, cholesterol, and DMG-PEG2000 at a ratio of about 40-60 to 5-15 to 31-45 to 0.05-2.

In embodiments, the second solution comprises TPGS1000 and SM-102.

In embodiments, the second solution comprises SM-102 and TPGS1000 at a ratio of about 62.5:37.5, about 55:45, about 70:30, about 80:20, or about 50:50. In embodiments, the second solution comprises SM-102 and TPGS1000 at a ratio of 62.5:37.5. In embodiments, the second solution comprises SM-102 and TPGS1000 at a ratio of about 50:50. In embodiments, the second solution comprises SM-102 and TPGS1000 at a ratio of about 70:30.

In embodiments, the ratio of ionizable cationic lipid to the helper lipids is about 1:3, about 1:2, about 1:1, about 2:1, or about 3:1. In embodiments, the ratio of KT-001 to the helper lipids is about 1:3, about 1:2, about 1:1, about 2:1, or about 3:1. In embodiments, the ratio of SM-102 to the helper lipids is about 1:3, about 1:2, about 1:1, about 2:1, or about 3:1.

In embodiments, the percentage of ionizable cationic lipid is about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%. In embodiments, the percentage of ionizable cationic lipid is about 25%, about 30%, about 35%, about 40%, about 45%, about 50%, about 55%, about 60%, about 65%, about 70%, or about 75%.

In embodiments, the third solution includes two or three species of lipid molecules, wherein the species of lipid molecules are selected from an ionizable cationic lipid species, a cationic lipid species, an anionic lipid species, a neutral lipid species, a helper lipid species, or any combination thereof.

In embodiments, the third solution comprises DSPC and cholesterol. In embodiments, the third solution comprises DSPC.

In embodiments, the third solution comprises a lipid mixture of DOTAP and DOPE. In embodiments, the third solution comprises a lipid mixture of SM-102, DOTMA, and DSPC. In embodiments, the third solution comprises a lipid mixture of BAE, DOTMA, and DSPC. In embodiments, the third solution comprises a lipid mixture of KT-001, DOTMA, and DSPC.

In embodiments, the third solution comprises DOTAP and DOPE at a ratio of about 1:3, about 1:2, about 1:1, about 2:1, about 3:1, about 4:1, about 5:1, or about 10:1. In embodiments, the third solution comprises DOTAP and DOPE at a ratio of about 3:1. In embodiments, the third solution comprises DOTAP and DOPE at a ratio of 3:1.

In embodiments, the third solution comprises SM-102, DOTMA, and DSPC in the range of about 40-80:1-20:2-40. In embodiments, the third solution comprises SM-102:DOTMA:DSPC at a ratio of 7:1:2.

In embodiments, the third solution comprises BAE, DOTMA, and DSPC in the range of about 60-120:1-20:2-40. In embodiments, the third solution comprises BAE, DOTMA, and DSPC in a ratio of 10.4:1:2.

In embodiments, the third solution comprises KT-001, DOTMA, and DSPC in the range of about 50-90:1-20:2-40. In embodiments, the third solution comprises KT-001, DOTMA, and DSPC in a ratio of 7.9:1:2.

Any lipid species may be mixed at any ratios suitable for encapsulating nanoparticles. In embodiments, a suitable lipid solution contains a mixture of desired lipids including ionizable cationic lipid species, a cationic lipid species, an anionic lipid species, a neutral lipid species, and/or a helper lipid species. In embodiments, a suitable lipid solution contains a mixture of desired lipids including one or more cationic lipids, one or more helper lipids, one or more anionic lipids, one or more ionizable cationic lipids, and one or more neutral lipids. In embodiments, a suitable lipid solution contains a mixture of desired lipids including one or more neutral lipids and one or more helper lipids. In embodiments, a suitable lipid solution contains one or more helper lipids.

### Cell Transfection

In an aspect, provided herein is a method for transfecting a cell comprising contacting a cell with the lipid-coated nanoparticle as described herein or the pharmaceutical composition as described herein, including in embodiments.

In embodiments, the transfection is in-vitro. In embodiments, the transfection is in-vivo.

In embodiments, provided herein is a method for administering a payload to a patient, comprising administering the lipid-coated nanoparticle as described herein or the pharmaceutical composition as described herein, including in embodiments, to a patient in need thereof. In embodiments, provided herein is a method for administering a payload to a patient, comprising administering the lipid-coated nanoparticle as described herein, including in embodiments, to a patient in need thereof. In embodiments, provided herein is a method for administering a payload to a patient, comprising administering the pharmaceutical composition as described herein, including in embodiments, to a patient in need thereof.

In embodiments, provided herein is a method for transfecting a cell with a payload, comprising contacting a cell with the lipid-coated nanoparticle as described herein or the pharmaceutical composition as described herein, including in embodiments. In embodiments, provided herein is a method for transfecting a cell with a payload, comprising contacting a cell with the lipid-coated nanoparticle as described herein, including in embodiments. In embodiments, provided herein is a method for transfecting a cell with a payload, comprising contacting a cell with the pharmaceutical composition as described herein, including in embodiments.

### EXAMPLES

As used herein, the symbols and conventions used in these processes, schemes and examples, regardless of whether a particular abbreviation is specifically defined, are consistent with those used in the contemporary scientific literature, for example, the Journal of the American Chemical Society, the Journal of Medicinal Chemistry, or the Journal of Biological Chemistry.

The following examples are meant to be illustrative and can be used to further understand embodiments of the present disclosure and should not be construed as limiting the scope of the present teachings in any way.

### Definitions of Abbreviations Used

ACN Acetonitrile
AcOH Acetic acid
aq. Aqueous
BAE or BAE-001 Ionizable cationic lipid (proprietary-U.S. provisional 63/313,648)

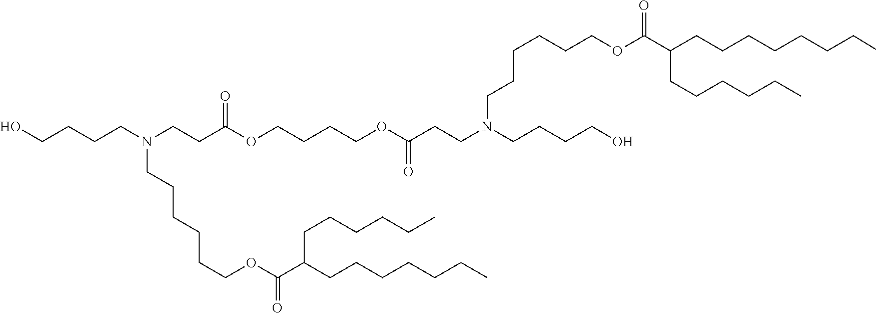 [SMILES: CCCCCCCCC(CCCCCC)C(=O)OCCCCCCN(CCCCO)CCC(=O)OCCCCOC(=O)CCN(CCCCO)CCCCCCOC(=O)C(CCCCCC)CCCCCCC]

CSA Camphorsulfonic acid
CSC12 Thiophosgene
D or Da Molecular weight unit (equivalent to g/mol)
DC-cholesterol (3β-[N-(N′,N′-dimethylaminoethane)-carbamoyl]cholesterol
DCM Dichloromethane
DOPE 1,2-Dioleoyl-sn-glycero-3-phosphoethanolamine
DOTAP 1,2-Dioleoyl-3-trimethylammonium propane
DOTMA 1,2-di-O-octadecenyl-3-trimethylammonium propane
DSPC 1,2-Distearoyl-sn-glycero-3-phosphocholine
EDC 1-Ethyl-3-(3-dimethylaminopropyl)carbodiimide
Et Ethyl
Eq Equivalents
g Gram(s)
GFP or GFP-mRNA mRNA encoding Green fluorescent protein (used interchangeably)
hr Hour (hours)
HCl Hydrochloric acid
HPLC High-performance liquid chromatography
KT-001 or KT001 Ionizable cationic lipid (proprietary-US provisional 63/313,648

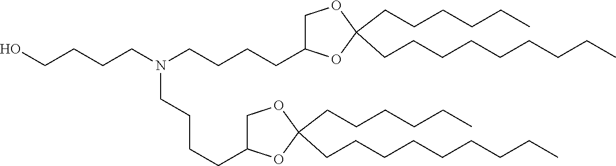 [SMILES: CCCCCCCCCC1(CCCCCC)OCC(CCCCN(CCCCO)CCCCC2COC(CCCCCC)(CCCCCCCCC)O2)O1]

LC/MS Liquid chromatography-mass spectrometry
Me Methyl
mg Milligrams
MeOH Methanol
mL Milliliter(s)
μL/μL Microliter(s)
mol Moles
mmol Millimoles
μmol/μmol Micromoles
MS Mass spectrometry
NaBH(OAc)₃ Sodium triacetoxyborohydride
PCC Pyridinium chlorochromate
PDI polydispersity index
PEG Polyethylene glycol
r.t. Room temperature
SM-102 1-Octylnonyl 8-[(hydroxyethyl)[6-oxo-6-(undecyloxy)hexyl]amino]octanoate
TBS buffer
Tris-buffered saline
TBS sucrose buffer Tris-sucrose buffer (Aydogan et al. 2014, Folia Microbiol 59:9-16)
TBT buffer Tris-trehalose buffer
TEA or Et3N Triethylamine
TFA Trifluoracetic acid
THF Tetrahydrofuran
TLC Thin Layer Chromatography

### Example 1: Preparation of Liposomes Containing LNPs Using Manual Mixing

Liposomes were prepared as follows: DOTAP (366.7 μL, 20 μg/μL in EtOH) and DOPE (130.2 μL, 20 μg/μL) were added to a 2 mL serum vial to make 496.9 μL of lipid solution (lipid concentration: 27 mM; molar ratio of lipids:DOTAP/DOPE=3/1). The lipid solution was drawn into a 1 mL BD syringe. Air bubbles were removed by gently tapping the syringe. Then, the syringe was loaded onto the Ignite NanoAssemblr cartridge. 2.48 mL of 2× Tyrode's Buffer was drawn into a 3 mL BD syringe. Air bubbles were removed by gently tapping the syringe. Then the syringe was loaded onto the Ignite NanoAssemblr cartridge. Flow rate was set at 12 mL/min with ratio of: 2× Tyrode's Buffer/Lipid solution=5/1 to produce liposome particles. The liposome particles were then collected into a dialysis bag (100 KD) and dialyzed with 2× Tyrode's buffer. The buffer was changed 3 times every 2 hours for a total of 6 hours.

LNPs were prepared as follows: 1. Lipid Solution: To a 5 mL Eppendorf tube was added KT-001 (550.5 μL, 20 mg/mL in EtOH), DSPC (105.4 μL, 20 mg/mL in EtOH), Cholesterol (198.7 μL, 20 mg/mL in EtOH), and DMG-PEG2000 (62.8 μL, 20 mg/mL in EtOH) and EtOH (1796 μL) to make 2.713 mL lipid solution (lipid concentration: 10 mM; molar ratio of lipids: KT-001/DSPC/Cholesterol/DMG-PEG2000=49.9/10/38.4/0.17).
2. mRNA solution: mRNA (600 μL, 1 mg/mL) was dissolved in sodium acetate buffer (pH 5, 25 mM) to make a mRNA sodium acetate solution (pH 5, 25 mM, mRNA 73.7 μg/mL).
3. TBS sucrose buffer preparation: Tris base (9.688 g, 0.08 mol), sucrose (320.00 g), and water for injection (WFI 3.8 L) were added to a 6L beaker. The mixture was allowed to stir at room temperature for 2 hours or until all materials dissolved. The pH of the solution was adjusted with HCl (1N) to pH 7.3 to 7.4. The overall volume of the solution was brought to 4 L, and the solution was sterile filtered using 0.22 μM filtration.
4. LNP formulation: mRNA solution (8.14 mL, 86.4 μg/mL) was drawn into a 10 mL BD syringe, air bubbles were removed by gently tapping the syringe. Then, the syringe was loaded onto the Ignite NanoAssemblr cartridge. Lipid solution (10 mM) was drawn into a 3 mL BD syringe, air bubbles were removed by gently tapping the syringe. Then, the syringe (with lipid solution) was loaded onto the Ignite NanoAssemblr cartridge. Flow rate was set at 12 mL/min, with ratio of the aqueous solution to EtOH solution as 3/1 (with 0.1 mL and 0.05 mL waste volume at the beginning and the ending stage). The LNPs were then collected into a dialysis bag (100 KD) for buffer exchange.
5. Dialysis: The dialysis bag with the LNPs was dialyzed with TBS sucrose buffer (1 L). The buffer was changed 3 times, every 6 hours. The size, and zeta were measured using the Zetasizer Ultra. The mRNA concentration and encapsulation rate were determined using Ribogreen-based mRNA assay using a plate reader (Quant-iT™ RiboGreen® RNA Reagent Assay Kit; Invitrogen Catalog #R11490, R11491, T11493). The mRNA concentration was 121.48 μg/mL and encapsulation rate was 84.8%.

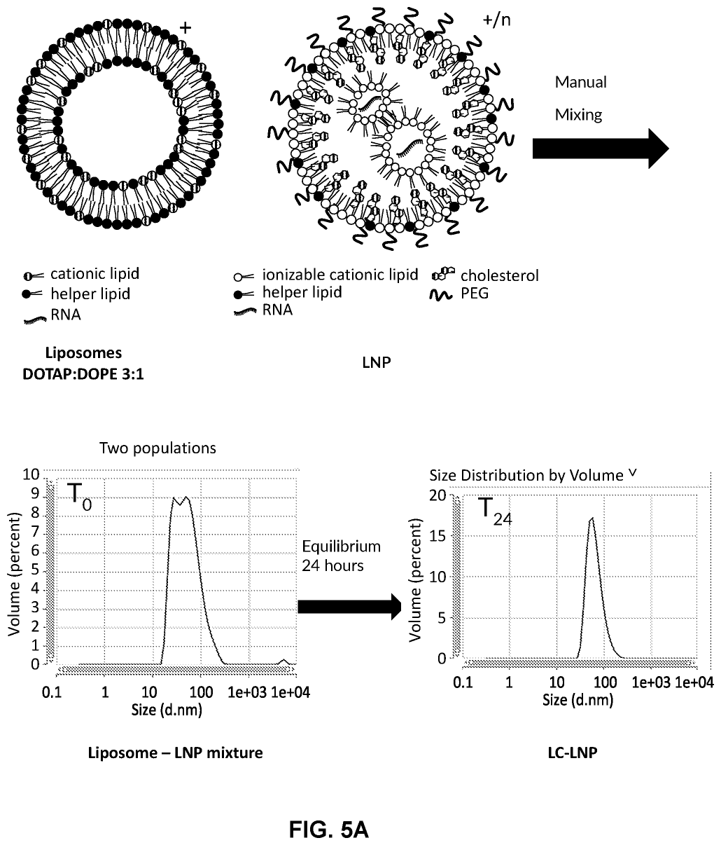

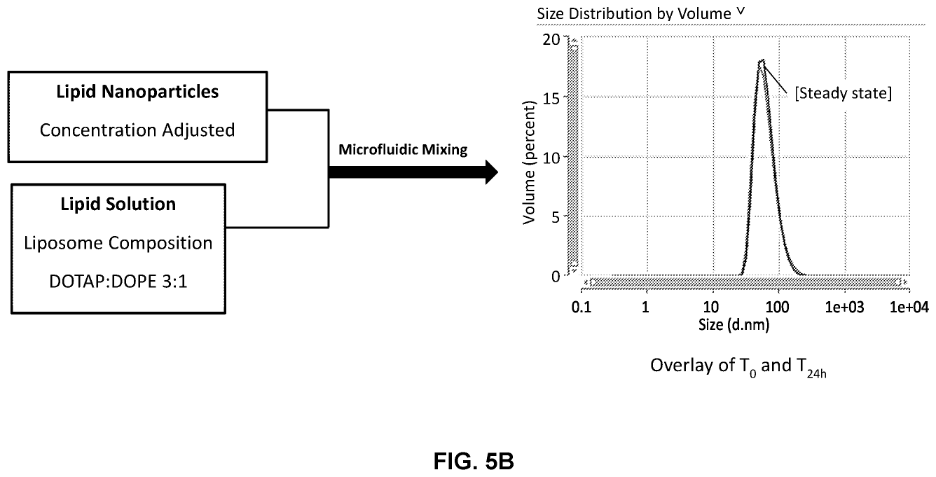

Lipid coated LNPs were prepared as follows: After dialysis, 200 μL of the DOTAP/DOPE=3/1 liposomes (prepared above) were added to 1000 μL of LNP solution (prepared above) (200 μg/mL mRNA) at a 1:5 ratio and measured for size and zeta using the Zetasizer Ultra to monitor particle stability and equilibrium. FIG. 5A shows that when liposomes and LNPs are mixed manually there are initially two populations of particles corresponding to the liposomes and the LNPs, but after 24 hours of mixing a single peak belonging to LC-LNPs is formed.

### Example 2: Preparation of Liposomes Containing LNPs Using Microfluidic Mixing

Lipid coating solution was prepared as follows: DOTAP (366.7 μL, 20 μg/μL in EtOH) and DOPE (130.2 μL, 20 μg/μL) were added to a 2 mL serum vial to make 496.9 μL of lipid solution (lipid concentration: 27 mM; molar ratio of lipids: DOTAP/DOPE=3/1).

LNPs were prepared as described above in Example 1. LNP solution was prepared as follows: LNP solution (1859.4 μL, 200 μg/mL mRNA) and 140.6 μL TBS sucrose buffer (pH 7.3-7.4) were added to a 5 mL Eppendorf tube to yield 2 mL of LNP solution (185.9 μg/mL mRNA).

Lipid coated LNPs were prepared as follows: The LNP solution (2 mL, 185.9 μg/mL mRNA) was drawn in a 5 mL BD syringe. Air bubbles were removed by gently tapping the syringe. Then the syringe was loaded onto the Ignite NanoAssemblr cartridge. The lipid solution (400 μL, 27 mM) was drawn into a 1 mL BD syringe. Air bubbles were removed by gently tapping the syringe. Then the syringe was loaded onto the Ignite NanoAssemblr cartridge. Flow rate was set at 12 mL/min, ratio of the aqueous solution to the EtOH solution as 5/1 (Concentration adjusted LNP/Lipid solution=5/1). The waste volumes for the beginning and ending stages were set to 0.1 mL and 0.05 mL respectively. The LC-LNP solution was collected into a dialysis bag (100 KD) for buffer exchange.

Dialysis: The dialysis bag with the LC-LNP solution was dialyzed with TBS sucrose buffer (1 L, pH 7.3-7.4). The buffer was changed every 3 hours for 9 hours. The size and zeta of LC-LNPs were measured by Zetasizer Ultra. The mRNA concentration and encapsulation rate were determined using Ribogreen-based mRNA assay using a plate reader (Quant-iT™ RiboGreen® RNA Reagent Assay Kit; Invitrogen Catalog #R11490, R11491, T11493). The mRNA concentration was 39.83 μg/mL and encapsulation rate was 84.05%.

FIG. 5B shows that when lipid solution and LNPs are mixed using microfluidic mixing there is an immediate formation of LC-LNPs. There is only a single peak indicating a single population of particles, immediately upon mixing and after 24 hours of mixing (the peaks lay on top of each other). This indicates that microfluidic mixing allows for an optimized, immediate, formation of LC-LNPs. This process is both faster and more scalable than the manual mixing process.

Characterization of Liposomes, LNPs, and LC-LNPs from Examples 1 and 2:

TABLE 1

| | |
|---|---|
| Size (nm) | Zeta Potential (mV) |

| | T₀ | T_{24 h} | T₀ | T_{24 h} |
|---|---|---|---|---|
| Liposome | 85.68 | N/A | 53.53 | N/A |
| (manual) | | | | |
| LNP | 91.13 | N/A | 7.76 | N/A |
| LC-LNP | 97.25 | 82.11 | 33.15 | 37.04 |
| (manual) | | | | |
| LC-LNP | 78.8 | 79.3 | 29.04 | 27.75 |
| microfluidic | | | | |

### Example 3: Preparation of Liposomes Containing Nanoemulsion Using Manual Mixing

Liposomes were prepared as described above in Example 1.

Nanoemulsion solution was prepared as follows: 750 mg of SM102, 450 mg of TPGS1000, and 1.5 mL EtOH were added to a 5 mL Eppendorf tube. The mixture was then sonicated for 10 minutes to produce a homogenous lipid solution. The lipid solution was then slowly added to 28.5 mL of 2λ Tyrode's buffer in a 50 mL centrifuge tube while being mixed by a high shear homogenizer at 10,000 rpm for 3 minutes. The solution was then added to the LM020 microfluidizer for 3 cycles at 15,000 psi to create an emulsion of SM102 (25 mg/mL), TPGS1000 (15 mg/mL), and 2× Tyrode's buffer. 1 mL of this emulsion (SM102 25 mg/mL, TPGS1000 15 mg/mL) was then diluted with 7.93 mL of 2× Tyrode's buffer to produce 8.93 mL of concentration adjusted emulsion (SM102 2.8 mg/mL, TPGS1000 1.68 mg/mL).

Lipid coated nanoemulsion was prepared as follows: After dialysis, 200 μL of DOTAP/DOPE=3/1 liposome was added to 1000 μL of emulsion solution (SM102 4.2 mg/mL) at a 1:5 ratio. The size and zeta of LC-nanoemulsion were measured by Zetasizer Ultra to monitor particle stability and equilibrium.

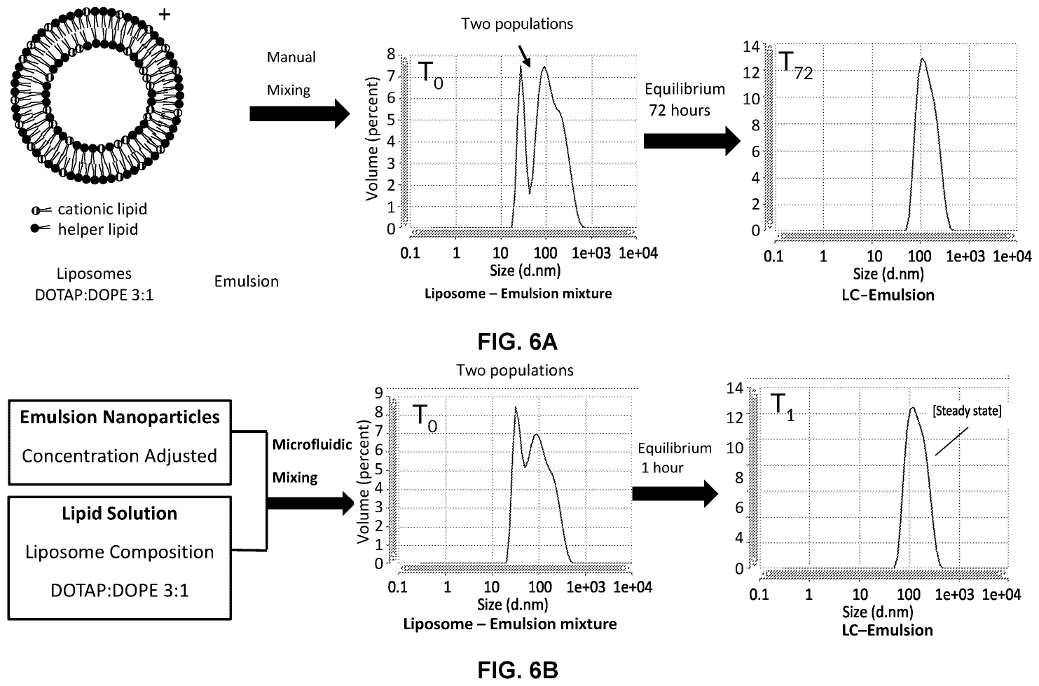

FIG. 6A shows that when liposomes and nanoemulsion are mixed manually there are initially two populations of particles corresponding to the liposomes and the nanoemulsion, but after 72 hours of mixing a single peak belonging to LC-nanoemulsion is formed.

### Example 4: Preparation of Liposomes Containing Nanoemulsion Using Microfluidic Mixing

Lipid coating solution was prepared as follows: DOTAP (366.7 μL, 20 μg/μL in EtOH) and DOPE (130.2 μL, 20 μg/μL) were added to a 2 mL serum vial to make 496.9 μL of lipid solution (lipid concentration: 27 mM; molar ratio of lipids: DOTAP/DOPE=3/1).

Nanoemulsion solution was prepared as described above in Example 3.

Lipid coated nanoemulsion was prepared as follows: 2 mL of concentration adjusted emulsion (SM102 2.8 mg/mL, TPGS1000 1.68 mg/mL) was drawn in a 5 mL BD syringe. Air bubbles were removed by gently tapping the syringe. Then the syringe was loaded onto the Ignite NanoAssemblr cartridge. The lipid solution (400 μL, 27 mM) was drawn into a 1 mL BD syringe. Air bubbles were removed by gently tapping the syringe. Then the syringe was loaded onto the Ignite NanoAssemblr cartridge. Flow rate was set at 12 mL/min, ratio of the aqueous solution to the EtOH solution as 5/1 (Concentration adjusted Emulsion/Lipid solution=5/1). The waste volumes for the beginning and ending stages were set to 0.1 mL and 0.05 mL respectively. The lipid coated nanoemulsion solution was collected into a dialysis bag (100 KD) for buffer exchange.

Dialysis: The dialysis bag with the LC-nanoemulsion solution was dialyzed with 2× Tyrode's buffer. The buffer was changed every 3 hours for 9 hours. The size and zeta of LC-nanoemulsion were measured by Zetasizer Ultra. The lipid concentrations were determined using UPLC.

FIG. 6B shows that when liposomes and nanoemulsion are mixed using microfluidic mixing there are initially two populations of particles corresponding to the liposomes and the nanoemulsion, but after 1 hour of mixing a single peak belonging to LC-nanoemulsion is formed.

Characterization of Liposomes, Nanoemulsion, and LC-LNPs from Examples 3 and 4:

TABLE 2

| Size (nm) | | | Zeta Potential (mV) | |
|---|---|---|---|---|
| | T₀ | T_{F} | T₀ | T_{F} |
| Liposome (manual) | 157.0 | N/A | 47.07 | N/A |
| Nanoemulsion | 133.3 | N/A | 5.50 | N/A |
| LC-nanoemulsion | 147.3 | 147.4 | 7.19 | 8.40 |
| (manual) | | T_{F} = 72 h | | T_{F} = 72 h |
| LC-nanoemulsion | 131.5 | 135.2 | 5.49 | 9.33 |
| microfluidic | | T_{F} = 1 h | | T_{F} = 1 h |

### Example 5: Preparation of Lipid Coated GFP-LNPs (LPX-U-GFP-LNP0607) Using Microfluidic Mixing

Lipid coating solution was prepared as follows: SM102 (280 μL, 20 mg/mL in ETOH), DOTMA (40 L, 20 mg/mL in ETOH) and DSPC (80 μL, 20 mg/mL in ETOH) were added to a 1 mL Eppendorf tube to make 400 μL of lipid solution (lipid concentration: 22.7 mM; molar ratio of lipids: SM102/DOTMA/DSPC-78.9/11.9/20.2).

LNPs were prepared as described above in Example 1, with GFP mRNA used in the procedure. GFP-LNP was formed with a concentration of 310.03 μg/mL.

LNP solution was prepared as follows: LNP solution (645.1 μL, 310.03 μg/mL GFP mRNA) and 354.9 μL TBS sucrose buffer (pH 7.3-7.4) were added to a 5 mL Eppendorf tube to yield 1 mL of LNP solution (200 μg/mL GFP mRNA).

Lipid coated LNPs were prepared as follows: The LNP solution (1 mL, 200 μg/mL GFP mRNA) was drawn in a 1 mL BD syringe. Air bubbles were removed by gently tapping the syringe. Then the syringe was loaded onto the Ignite NanoAssemblr cartridge. The lipid solution (200 μL, 22.7 mM, N/P 7.49) was drawn into a 1 mL BD syringe. Air bubbles were removed by gently tapping the syringe. Then the syringe was loaded onto the Ignite NanoAssemblr cartridge. Flow rate was set at 12 mL/min, ratio of the aqueous solution to the EtOH solution as 5/1. The waste volumes for the beginning and ending stages were set to 0.05 mL. The LC-LNP solution was collected into a dialysis bag (50 KD) for buffer exchange.

Dialysis: The dialysis bag with the LC-LNP solution was dialyzed with TBS sucrose buffer (1 L, pH 7.3-7.4) one time. The size and zeta of LC-LNPs were measured by Zetasizer Ultra. The GFP mRNA concentration, encapsulation rate was determined using Ribogreen-based mRNA assay using plate reader (Quant-iT™ RiboGreen® RNA Reagent Assay Kit; Invitrogen Catalog #R11490, R11491, T11493). The mRNA concentration was 151.9 μg/mL and encapsulation rate was 75.9%.

### Example 6: Preparation of Lipid Coated GFP-LNPs (BAE-LPX-U-GFP (KT001)-LNP) Using Microfluidic Mixing

Lipid coating solution was prepared as follows: BAE (207.7 μL, 20 mg/mL in ETOH), DOTMA (20 μL, 20 mg/mL in ETOH) and DSPC (40 μL, 20 mg/mL in ETOH) were added to a 1 mL eppendorf tube to make 267.7 μL of lipid solution (lipid concentration: 17 mM; molar ratio of lipids: BAE/DOTMA/DSPC=78.9/11.9/20.2).

LNPs were prepared as described above in Example 1, with GFP mRNA used in the procedure. GFP-LNP was formed with a concentration of 258.9 μg/mL.

LNP solution was prepared as follows: LNP solution (700 μL, 258.9 μg/mL GFP mRNA) and 517 μL of water for injection (WFI) were added to a 5 mL Eppendorf tube to yield 1.217 mL of LNP solution (149 μg/mL GFP mRNA).

Lipid coated LNPs were prepared as follows: The LNP solution (1 mL, 149 μg/mL GFP mRNA) was drawn in a 1 mL BD syringe. Air bubbles were removed by gently tapping the syringe. Then the syringe was loaded onto the Ignite NanoAssemblr cartridge. The lipid solution (200 μL, 17 mM, N/P 7.5) was drawn into a 1 mL BD syringe. Air bubbles were removed by gently tapping the syringe. Then the syringe was loaded onto the Ignite NanoAssemblr cartridge. Flow rate was set at 12 mL/min, ratio of the aqueous solution to the EtOH solution as 5/1. The waste volumes for the beginning and ending stages were set to 0.05 mL. The LC-LNP solution was collected into a dialysis bag (50 KD) for buffer exchange.

Dialysis: The dialysis bag with the LC-LNP solution was dialyzed with TBS sucrose buffer (1 L, pH 7.3-7.4) one time. The size and zeta of LC-LNPs were measured by Zetasizer Ultra. The GFP mRNA concentration, encapsulation rate was determined using Ribogreen-based mRNA assay using plate reader (Quant-iT™ RiboGreen® RNA Reagent Assay Kit; Invitrogen Catalog #R11490, R11491, T11493). The mRNA concentration was 196.8 μg/mL and encapsulation rate was 86.0%.

### Example 7: Preparation of Lipid Coated GFP-LNPs (KT001-LPX-U-GFP (KT001)-LNP) Using Microfluidic Mixing

Lipid coating solution was prepared as follows: KT001 (201.4 μL, 20 mg/mL in ETOH), DOTMA (26.7 μL, 20 mg/mL in ETOH) and DSPC (53.3 μL, 20 mg/mL in ETOH) were added to a 1 mL eppendorf tube to make 290.4 μL of lipid solution (lipid concentration: 21.5 mM; molar ratio of lipids: KT001/DOTMA/DSPC=78.9/11.9/20.2).

LNPs were prepared as described above in Example 1, with GFP mRNA used in the procedure. GFP-LNP was formed with a concentration of 258.9 g/mL.

LNP solution was prepared as follows: LNP solution (700 μL, 258.9 μg/mL GFP mRNA) and 300 μL of water for injection (WFI) were added to a 5 mL Eppendorf tube to yield 1 mL of LNP solution (189 μg/mL GFP mRNA).

Lipid coated LNPs were prepared as follows: The LNP solution (1 mL, 189 μg/mL GFP mRNA) was drawn in a 1 mL BD syringe. Air bubbles were removed by gently tapping the syringe. Then the syringe was loaded onto the Ignite NanoAssemblr cartridge. The lipid solution (180 μL, 21.5 mM, N/P 7.5) was drawn into a 1 mL BD syringe. Air bubbles were removed by gently tapping the syringe. Then the syringe was loaded onto the Ignite NanoAssemblr cartridge. Flow rate was set at 12 mL/min, ratio of the aqueous solution to the EtOH solution as 5/1. The waste volumes for the beginning and ending stages were set to 0.05 mL. The LC-LNP solution was collected into a dialysis bag (50 KD) for buffer exchange.

Dialysis: The dialysis bag with the LC-LNP solution was dialyzed with TBS sucrose buffer (1 L, pH 7.3-7.4) one time. The size and zeta of LC-LNPs were measured by Zetasizer Ultra. The GFP mRNA concentration, encapsulation rate was determined using Ribogreen-based mRNA assay using plate reader (Quant-iT™ RiboGreen® RNA Reagent Assay Kit; Invitrogen Catalog #R11490, R11491, T11493). The mRNA concentration was 350.7 μg/mL and encapsulation rate was 80.7%.

Characterization of LNPs and LC-LNPs from Examples 5, 6, and 7:

TABLE 3

| | Size (nm)/PDI | Zeta Potential (mV) |
|---|---|---|
| LNP (Exp. 5) | 70.58/0.071 | −2.64 |
| LC-LNP (Exp. 5) | 87.71/0.075 | 0.732 |
| LNP (Exp. 6) | 82.91/0.080 | −0.655 |
| LC-LNP (Exp. 6) | 116.4/0.102 | 0.152 |
| LNP (Exp. 7) | 82.91/0.080 | −0.655 |
| LC-LNP (Exp. 7) | 100.0/0.120 | −0.11 |

### Example 8: Determination of Transfection Efficiency and Cell Viability of LPX-U-GFP-LNP0607 in Hek 293 Cells

Cells used for transfection efficiency and cell viability: P6 Hek293 cells, cell viability 97%. Cells were prepared for transfection as follows: Cells were gently pipetted to mix. 10 μl aliquot of cell suspension was used to perform Trypan Blue exclusion cell count using automatic cell counter to determine cell concentration and viability. Total number of cells was calculated. Cells were transferred to 50 ml centrifuge tube and spun at 1200 rpm or 250 g for 5 minutes. Old medium was discarded, and cells were resuspended in fresh completed medium (DMEM with 10% FBS). Cell concentration was adjusted to 0.4×10⁶ cells/ml.

Transfection conditions: 24 well cell culture plate was used. Cells were plated 0.2×10⁶ cells/well (i.e. 500 μl) and incubated overnight at 37° C. and 5% CO₂. After overnight incubation, the medium was changed to fresh completed medium (DMEM with 10% FBS). Lipid-coated LNPs (LPX-U-GFP-LNP0607) were added at 0.5, 1, or 2 μg/well. The plate was then incubated at 37° C. and 5% CO₂ for 24 hours. Flow cytometry was carried out after the 24-hour incubation to determine transfection efficiency and Trypan Blue exclusion cell count using automatic cell counter was used to determine cell viability. Lipofectamine Messenger MAX+mRNA (at the same concentration as in the LC-LNP) was used as positive control. Hek293 cells without LC-LNP were used as a negative control.

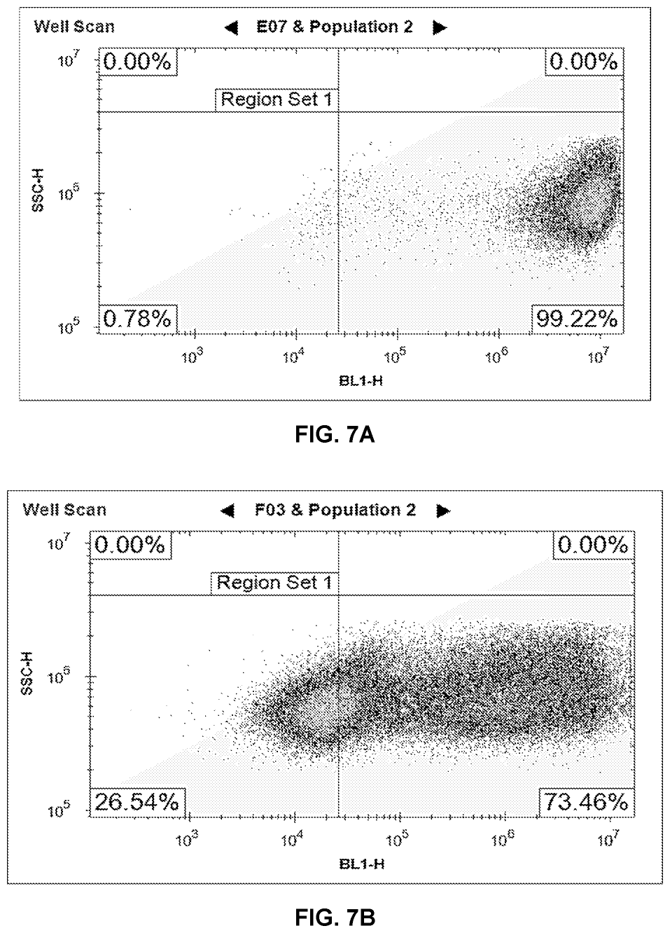

Scatterplots shown in FIG. 7A and FIG. 7B demonstrate that transfection efficiency of LPX-U-GFP-LNP0607 is much higher than that of the positive control Lipofectamine Messenger MAX.

### Example 9: Determination of Transfection Efficiency and Cell Viability of LPX-U-GFP-LNP0607 in Jurkat Cells

Cells used for transfection efficiency and cell viability: P6 Jurkat cells, cell viability 98%. Cells were prepared for transfection as follows: Cells were gently pipetted to mix. 10 μl aliquot of cell suspension was used to perform Trypan Blue exclusion cell count using automatic cell counter to determine cell concentration and viability. Total number of cells was calculated. Cells were transferred to 50 ml centrifuge tube and spun at 250 g for 5 minutes. Old medium was discarded, and cells were resuspended in fresh RPMI serum reduced medium (SRM-RPMI 1640). Cell concentration was adjusted to 1.0×10⁶ cells/ml.

Transfection conditions: 24 well cell culture plate was used. Cells were plated 5.0×10⁵/well (i.e. 500 μl). Then lipid-coated LNPs (LPX-U-GFP-LNP0607) were added at 1 or 3 μg/well and the plate was incubated for 4 hours at 37° C. and 5% CO₂. The plate was centrifuged at 250 g for 5 minutes and the old medium was discarded. 500 μl of Jurkat completed medium (RPMI 1640 with 10% FBS) was added to each well, and the plate was incubated at 37° C. and 5% CO₂ for 24 hours. Flow cytometry was carried out after the 24-hour incubation to determine transfection efficiency and Trypan Blue exclusion cell count using automatic cell counter was used to determine cell viability. Lipofectamine Messenger MAX+mRNA (at the same concentration as in the LC-LNP) was used as positive control. Jurkat cells without LC-LNP were used as a negative control.

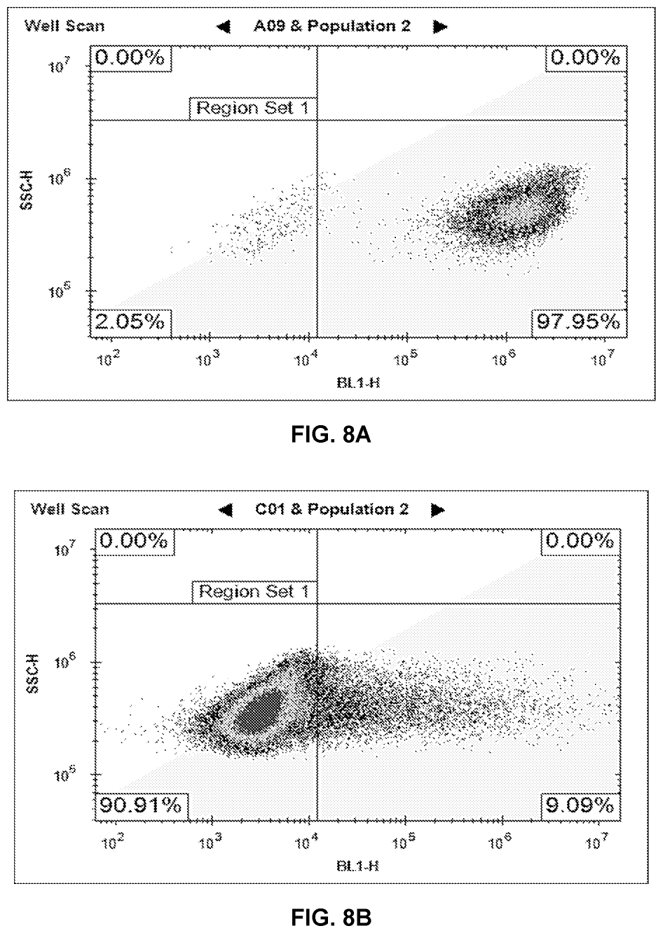

Scatterplots shown in FIG. 8A and FIG. 8B demonstrate that transfection efficiency of LPX-U-GFP-LNP0607 is more than 10 times higher than that of the positive control Lipofectamine Messenger MAX.

Transfection Efficiency and Cell Viability of LPX-U-GFP-LNP0607 from Examples 8 and 9:

TABLE 4

| | Transfection | Cell | |
|---|---|---|---|
| | Efficiency | Viability | Cell |
| Formulation | (%) | (%) | type |
| LPX-U-GFP-LNP0607 1 μg | 99.22 | 70 | Hek293 |
| Lipofectamine Messenger MAX 1 μg | 73.46 | 91 | Hek293 |
| LPX-U-GFP-LNP0607 1 μg | 97.95 | 73 | Jurkat |
| Lipofectamine Messenger MAX 1 μg | 9.09 | 93 | Jurkat |

### Example 10: Determination of Transfection Efficiency and Cell Viability of BAE-LPX-U-GFP (KT001)-LNP in Primary T Cells

Cells used for transfection efficiency and cell viability: T cells were isolated from donor blood which was ordered from Charles River with SEPAX-c pro kit and frozen in Cryostor cell cryopreservation media, CS10-Sigma c2874 at 2×10⁷/1 ml and stored at −200° C. Frozen activated primary T cells (2×10⁷) were thawed quickly in a bead bath and cell suspension was transferred into 15 ml tube with 10 ml complete T cell media (IL CTS OpTmizer T cell Expansion SFM with 1% CTS Glutamax, 5% CTS Immune Cell SR, IL2 300 IU/ml). The cells were centrifuged at 200 g for 10 minutes and after discarding the media the cells were resuspended in 10 ml of complete T cell media. 10 μl aliquot of cell suspension was used to perform Trypan Blue exclusion cell count using automatic cell counter to determine cell concentration and viability. Cell concentration was adjusted to 1.0×10⁶ cells/ml in complete T cell media. The cells were incubated at 37° C. and 5% CO₂ and cell growth was monitored. After 48 hours, cell medium was changed. Trypan Blue exclusion cell count using automatic cell counter to determine cell concentration and viability, cell viability was 92%.

Transfection conditions: 24 well cell culture plate was used. Cell concentration was adjusted to 1.0×10⁶ cells/ml with complete T cell media. Cells were plated 1.0×10⁶ cells/well (i.e. 1000 μl). Then lipid-coated LNPs (BAE-LPX-U-GFP (KT001)-LNP) were added at 1 or 3 μg/well, and the plate was incubated at 37° C. for 24 hours. Flow cytometry was carried out after the 24-hour incubation to determine transfection efficiency and Trypan Blue exclusion cell count using automatic cell counter was used to determine cell viability. Lipofectamine Messenger MAX+mRNA (at the same concentration as in the LC-LNP) was used as positive control. T cells without LC-LNP were used as a negative control.

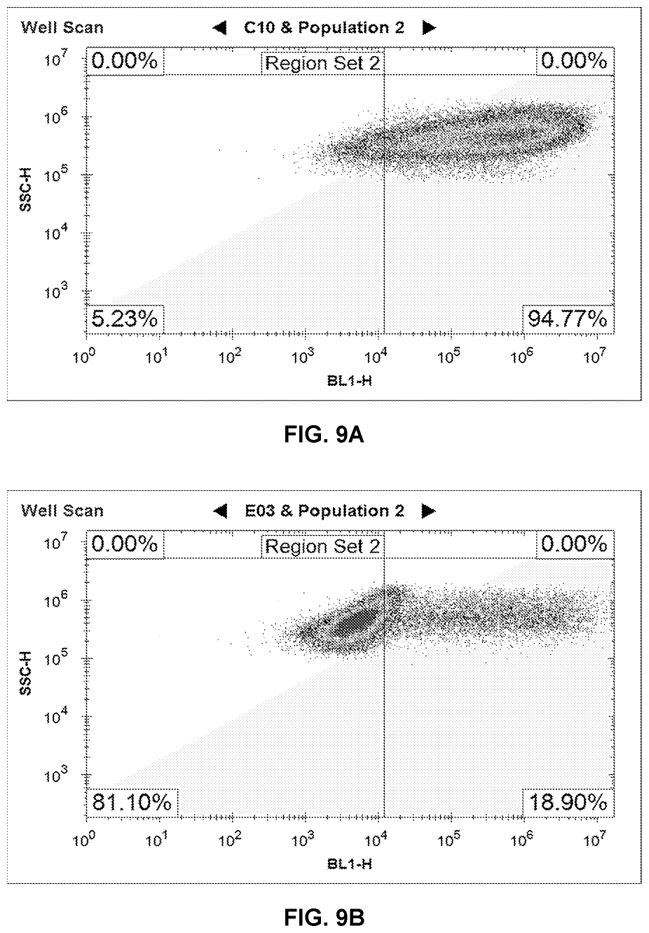

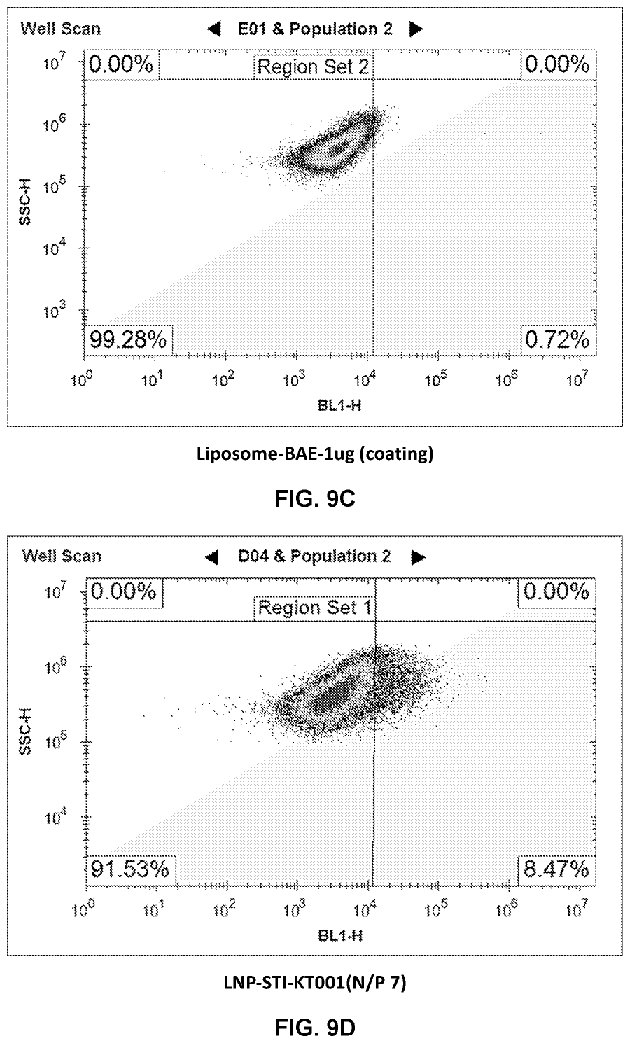

Scatterplots shown in FIG. 9A and FIG. 9B demonstrate that transfection efficiency of BAE-LPX-U-GFP (KT001)-LNP is much higher than that of the positive control Lipofectamine Messenger MAX.

Transfection efficiency was determined for two additional controls—the individual components of BAE-LPX-U-GFP (KT001)-LNP-Scatterplot in FIG. 9C shows transfection efficiency of the lipid coating alone (liposome-BAE) (BAE/DOTMA/DSPC=78.9/11.9/20.2), and Scatterplot shown in FIG. 9D shows transfection efficiency of the LNP alone (GFP(KT001)-LNP). Both scatterplots reveal a very low transfection efficiency for the separate components of lipid-coated lipid nanoparticles.

Transfection Efficiency and Cell Viability of BAE-LPX-U-GFP (KT001)-LNP from Example 10:

TABLE 5

| | Transfection | Cell | |
|---|---|---|---|
| | Efficiency | Viability | Cell |
| Formulation | (%) | (%) | type |
| BAE-LPX-U-GFP(KT001)- | 94.77 | 75 | Primary T Cell |
| LNP 1 μg | | | |
| Lipofectamine Messenger | 18.90 | 88 | Primary T Cell |
| MAX 1 μg | | | |
| BAE/DOTMA/DSPC coating | 0.72 | 90 | Primary T Cell |
| 1 μg | | | |
| GFP(KT001)-LNP lipid | 8.47 | 78 | Primary T Cell |
| nanoparticle 1 μg | | | |

### Example 11: Determination of Transfection Efficiency and Cell Viability of KT-001-LPX-U-GFP (KT001)-LNP in Primary T Cells

Cells used for transfection efficiency and cell viability: Frozen activated primary T cells cultured for 48 hours, cell viability 92%.

Transfection conditions were as described in Example 10.

Transfection Efficiency and Cell Viability of KT-001-LPX-U-GFP (KT001)-LNP from Example 11:

TABLE 6

| | Transfection | Cell | |
|---|---|---|---|
| | Efficiency | Viability | Cell |
| Formulation | (%) | (%) | type |
| KT-001-LPX-U- | 75.31 | 83 | Primary T Cell |
| GFP(KT001)-LNP 1 μg | | | |
| KT001-GFP-LNP lipid | 10.02 | 82 | Primary T Cell |
| nanoparticle 1 μg | | | |

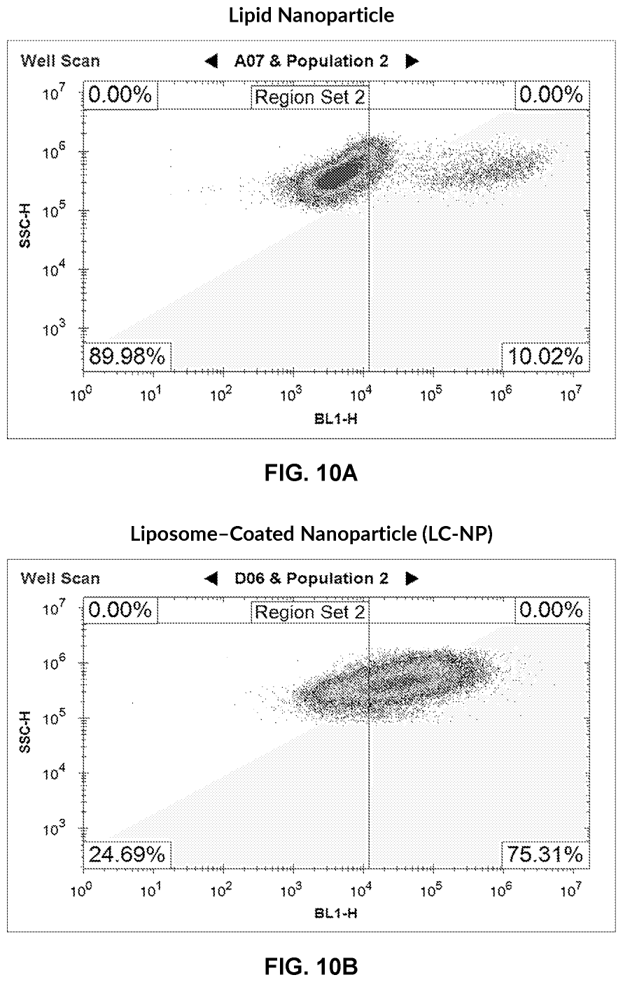

Transfection efficiency was determined for KT-001-LPX-U-GFP (KT001)-LNP (lipid-coated LNP) and for the KT001-GFP-LNP lipid nanoparticle alone. The LNP and LC-LNP have same lipid composition of LNP. Scatterplots in FIG. 10A and FIG. 10B show that despite identical inner core compositions, the LC-LNP and LNP have very different transfection efficiencies, with LC-LNP having 7-8 times higher transfection efficiency than LNP. In other words, LC-LNP has much higher potency than LNP, where the LNPs have the same composition. Thus, the potency of LC-LNP is a result of the lipid coating on top of the LNP.

### Example 12: In Vivo Study of Lipid-Coated Lipid Nanoparticles (LC-LNPs)

Preparation of KT001-LPX—U-Luc-LNP (KT001 Only) (rsF548)

Lipid coating solution was prepared as follows: KT001 (201.4 μL, 20 mg/mL in ETOH), DOTMA (26.7 μL, 20 mg/mL in ETOH) and DSPC (53.3 μL, 20 mg/mL in ETOH) were added to a 1 mL eppendorf tube to make 290.4 μL of lipid solution (lipid concentration: 21.5 mM; molar ratio of lipids: KT001/DOTMA/DSPC=78.9/11.9/20.2).

LNPs were prepared as described above in Example 1, with Luciferase (Luc) mRNA used in the procedure. Luc-LNP was formed with a concentration of 258.9 μg/mL.

LNP solution was prepared as follows: LNP solution (700 μL, 258.9 μg/mL Luc mRNA) and 300 μL of TBS sucrose buffer were added to a 5 mL Eppendorf tube to yield 1 mL of LNP solution (189 g/mL Luc mRNA).

Lipid coated LNPs were prepared as follows: The LNP solution (1 mL, 189 g/mL Luc mRNA) was drawn in a 5 mL BD syringe. Air bubbles were removed by gently tapping the syringe. Then the syringe was loaded onto the Ignite NanoAssemblr cartridge. The lipid solution (200 μL, 21.5 mM) was drawn into a 1 mL BD syringe. Air bubbles were removed by gently tapping the syringe. Then the syringe was loaded onto the Ignite NanoAssemblr cartridge. Flow rate was set at 12 mL/min, ratio of the aqueous solution to the EtOH solution as 5/1. The waste volumes for the beginning and ending stages were set to 0.05 mL. The LC-LNP solution was collected into a dialysis bag (50 KD) for buffer exchange.

Dialysis: The dialysis bag with the LC-LNP solution was dialyzed with TBS sucrose buffer (1 L, pH 7.3-7.4) one time. The size and zeta of LC-LNPs were measured by Zetasizer Ultra. The Luc mRNA concentration, encapsulation rate was determined using Ribogreen-based mRNA assay using plate reader (Quant-iT™ RiboGreen® RNA Reagent Assay Kit; Invitrogen Catalog #R11490, R11491, T11493). The mRNA concentration was 71.02 μg/mL and encapsulation rate was 74.4%.

Preparation of KT001-LPX—U-Luc-LNP (EggPA/DSPC) (rsF549)

Lipid coating solution was prepared as follows: KT001 (302.1 μL, 10 mg/mL in ETOH), EggPA (42.1 μL, 10 mg/mL in ETOH) and DSPC (80.0 μL, 10 mg/mL in ETOH) were added to a 1 mL eppendorf tube to make 424.2 μL of lipid solution (lipid concentration: 10.7 mM; molar ratio of lipids: KT001/EggPA/DSPC=78.9/11.9/20.2).

LNPs were prepared as described above in Example 1, with Luciferase (Luc) mRNA used in the procedure. Luc-LNP was formed with a concentration of 258.9 μg/mL.

LNP solution was prepared as follows: LNP solution (700 μL, 258.9 μg/mL Luc mRNA) and 300 μL of TBS sucrose buffer were added to a 5 mL Eppendorf tube to yield 1 mL of LNP solution (94 μg/mL Luc mRNA).

Lipid coated LNPs were prepared as follows: The LNP solution (1 mL, 94 μg/mL Luc mRNA) was drawn in a 5 mL BD syringe. Air bubbles were removed by gently tapping the syringe. Then the syringe was loaded onto the Ignite NanoAssemblr cartridge. The lipid solution (200 μL, 10.7 mM) was drawn into a 1 mL BD syringe. Air bubbles were removed by gently tapping the syringe. Then the syringe was loaded onto the Ignite NanoAssemblr cartridge. Flow rate was set at 12 mL/min, ratio of the aqueous solution to the EtOH solution as 5/1. The waste volumes for the beginning and ending stages were set to 0.05 mL. The LC-LNP solution was collected into a dialysis bag (50 KD) for buffer exchange.

Dialysis: The dialysis bag with the LC-LNP solution was dialyzed with TBS sucrose buffer (1 L, pH 7.3-7.4) one time. The size and zeta of LC-LNPs were measured by Zetasizer Ultra. The Luc mRNA concentration, encapsulation rate was determined using Ribogreen-based mRNA assay using plate reader (Quant-iT™ RiboGreen® RNA Reagent Assay Kit; Invitrogen Catalog #R11490, R11491, T11493). The mRNA concentration was 117.5 μg/mL and encapsulation rate was 80.3%.

Characterization of LC-LNPs (rsF548 and rsF549)

TABLE 7

| | | | Zeta Potential |
|---|---|---|---|
| LC-LNP | Composition | Size (nm)/PDI | (mV) |
| rsF548 | KT001-LPX-U-Luc-LNP | 96.57/0.06309 | −0.026 |
| | (KT001 only) | | |
| rsF549 | KT001-LPX-U-Luc-LNP | 121.6/0.146 | 3.291 |
| | (EggPA/DSPC) | | |

rsF548 and rsF549 share the same LNP core but have different lipid coating composition. Though both have KT001 and DSPC as part of their lipid coating, rsF548 includes DOTMA (cationic lipid) as its third lipid component whereas rsF549 includes EggPA (anionic lipid) as its third lipid component.

In vivo procedure: four mice were used in each test arm. rsF548 or rsF549 (concentration: 1 μg Luc mRNA in 100 μL saline) were administered per mouse to both quadriceps (50 μL per quadricep). At 3, 6, 24, and 48 hours post-injection of the LC-LNP, luciferin (Perkin Elmer, Shelton, CT) reconstituted in sterile PBS (final concentration of 15 mg/ml) was administered intraperitoneally into each mouse at a dose of 150 mg/kg body weight. The mice were anesthetized and placed on the imaging stage of the IVIS imaging system in the ventral position. Images were obtained 10 minutes after luciferin injection using the IVIS Imaging System (Perkin Elmer). Photons emitted from the mouse liver and muscle were quantified using Living Image Software (Perkin Elmer). Mean and SEM of Flux (photons/second), and corresponding AUC (total Flux over time) were calculated for comparison purposes.

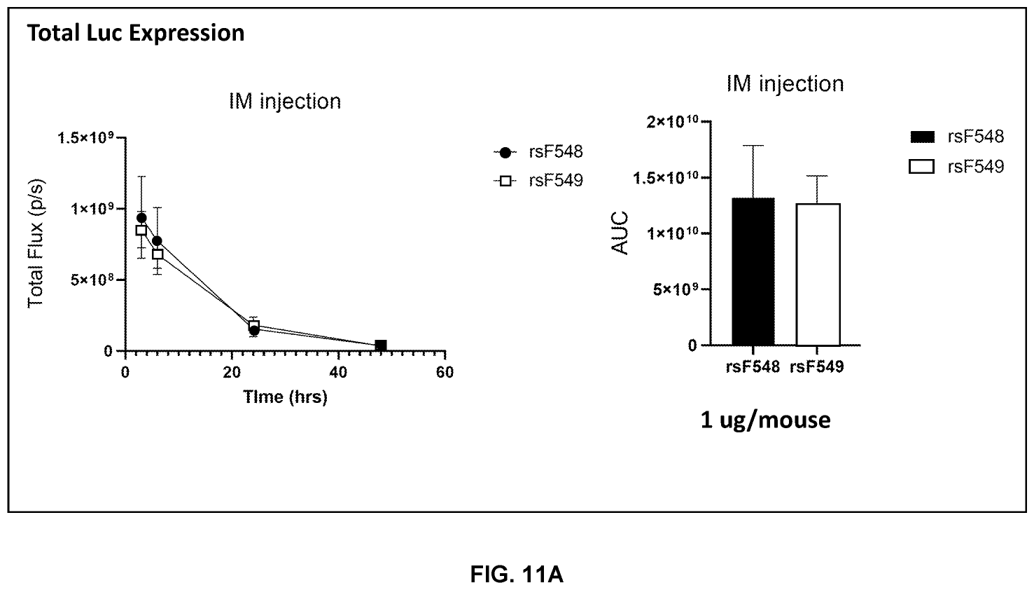

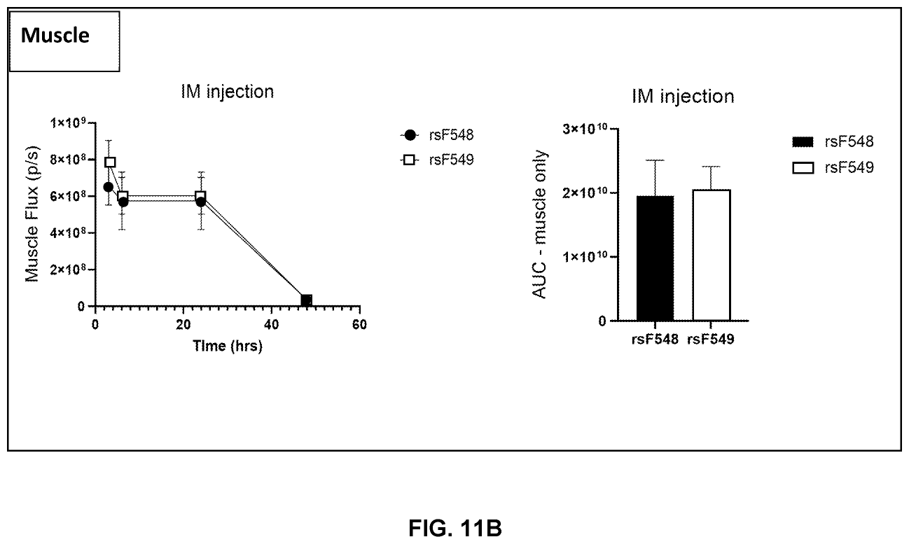

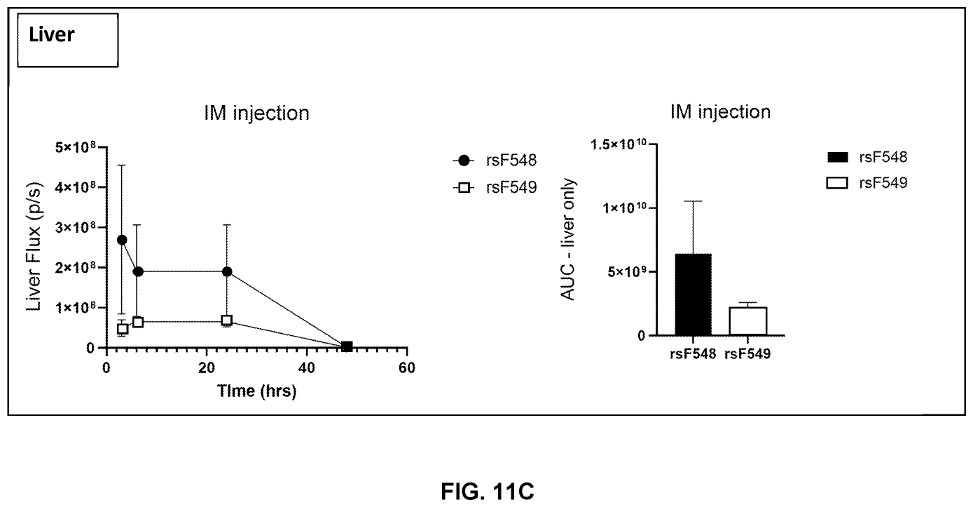

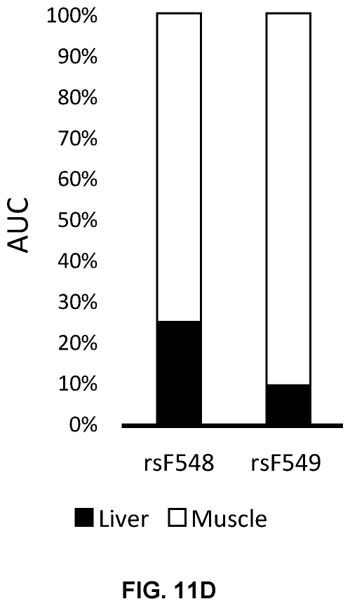

FIG. 11A shows that the total Luc expression levels of rsF548 and rsF549 are essentially the same. FIG. 11B shows that the muscle Luc expression levels of rsF548 and rsF549 are also essentially the same. In contrast, FIG. 11C shows that the liver Luc expression levels of rsF548 and rsF549 are significantly different. The relative Luc expression levels in liver and muscle for rsF548 and rsF549 are shown in FIG. 11D. These results indicate that it is possible to control off target drug delivery by controlling the surface coating of LC-LNPs. Thus, drug delivery can be restricted to desired locations.

### SYNTHETIC EXAMPLES

### Example S1: Synthesis of Compound KT-001

Compound KT-001 was prepared as shown in Scheme 1 below.

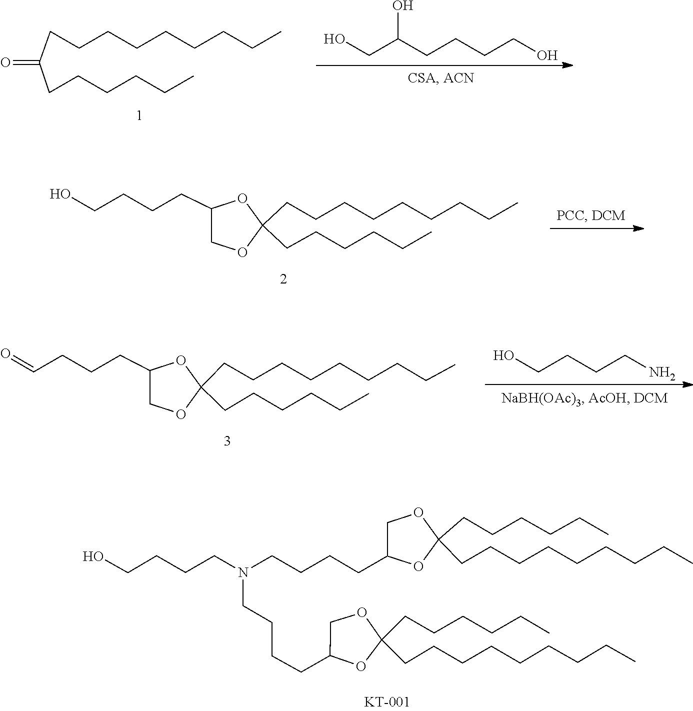 [SMILES: CCCCCCCCCC(=O)CCCCCC ; OCCCCC(O)CO ; CCCCCCCCCC1(CCCCCC)OCC(CCCCO)O1 ; CCCCCCCCCC1(CCCCCC)OCC(CCCC=O)O1 ; NCCCCO ; CCCCCCCCCC1(CCCCCC)OCC(CCCCN(CCCCO)CCCCC2COC(CCCCCC)(CCCCCCCCC)O2)O1]

Compound 2. To a mixture of compound 1 (1.00 g, 4.16 mmol) and 1,2,6-hexanetriol (1.12 g, 8.32 mmol) in anhydrous acetonitrile (20 mL) was added camphorsulfonic acid (CSA) (290 mg, 1.25 mmol). The resulting solution was refluxed for 16 hr until TLC indicated completion of reaction. Compound 2 (650 mg) was obtained by silica gel chromatography.

Compound 3. Pyridinium chlorochromate (PCC) (785 mg, 3.64 mmol) was added to a solution of compound 2 (650 mg, 1.82 mmol) in dichloromethane (30 mL). The reaction was stirred at room temperature for 2 hr until TLC indicated completion of reaction. Compound 3 (320 mg) was obtained by silica gel chromatography.

Compound KT-001. Acetic acid (4 μL, 0.068 mmol) was added to a mixture of compound 3 (300 mg, 0.846 mmol) and 4-amino-1-butanol (30 mg, 0.338 mmol) in dichloromethane (5 mL). The resulting mixture was stirred at r.t. for 15 min followed by addition of sodium triacetoxyborohydride (NaBH(OAc)₃) (215 mg, 1.01 mmol) and stirred at r.t. for another 4 h. Compound KT-001 (165 mg) was obtained by silica gel chromatography. ¹HNMR (500 MHZ, CDCl₃): δ=0.85-0.91 (m, 12H), 1.20-1.44 (m, 48H), 1.46-1.72 (m, 20H), 2.31-2.61 (br, 6H), 3.40-3.47 (m, 2H), 3.52-3.60 (m, 2H), 3.99-4.07 (m, 4H).

### Example S2: Synthesis of Compound BAE-001

Compound BAE-001 was prepared as shown in Scheme 2 below.

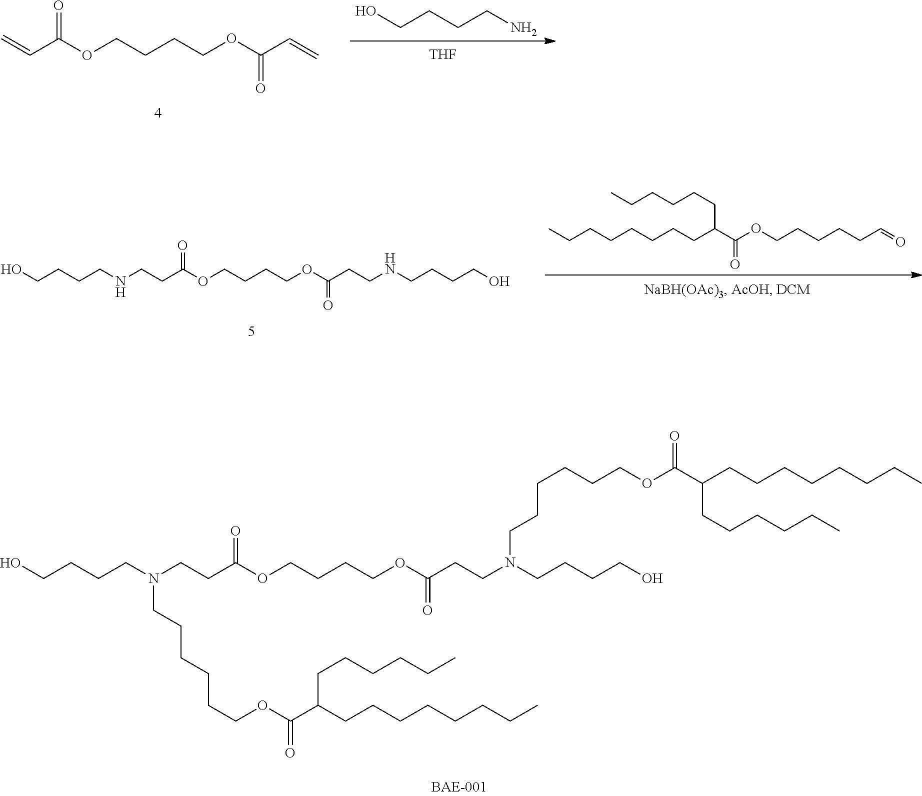 [SMILES: C=CC(=O)OCCCCOC(=O)C=C ; NCCCCO ; O=C(CCNCCCCO)OCCCCOC(=O)CCNCCCCO ; CCCCCCCCC(CCCCCC)C(=O)OCCCCCC=O ; CCCCCCCCC(CCCCCC)C(=O)OCCCCCCN(CCCCO)CCC(=O)OCCCCOC(=O)CCN(CCCCO)CCCCCCOC(=O)C(CCCCCC)CCCCCCCC ; "THF"]

4-amino-1-butanol (450 mg, 5.04 mmol) was added to the solution of compound 4 (500 mg, 2.52 mmol) in anhydrous THF (10 mL) and the resulting mixture was stirred at r.t. for 16 hr. Compound 5 (658 mg) was obtained by silica gel chromatography.

AcOH (5 μL, 0.08 mmol) was added to the mixture of compound 5 (154 mg, 0.41 mmol) and 6-(2′-hexyldecanoyloxy) hexanal (362 mg, 1.02 mmol) in DCM (10 mL) and the resulting solution was stirred at r.t. for 15 min. After addition of NaBH(OAc)₃ (260 mg, 1.23 mmol) the stirring was continued for another 4 hr until TLC indicated completion of reaction. Compound BAE-001 (98 mg) was obtained by silica gel chromatography.

¹HNMR (500 MHz, CDCl₃): δ=0.87 (t, 12H, J=7.0 Hz), 1.17-1.80 (m, 76H), 2.27-2.34 (m, 2H), 2.36-2.62 (br, 12H), 2.75-2.97 (m, 4H), 3.52-3.61 (m, 4H), 4.05 (t, 4H, J=6.5 Hz), 4.08-4.15 (m, 4H).

The complete disclosures of all publications cited herein are incorporated herein by reference in their entireties as if each were individually set forth in full herein and incorporated.

Various modifications and alterations to the embodiments disclosed herein will become apparent to those skilled in the art without departing from the scope and spirit of this disclosure. Illustrative embodiments and examples are provided as examples only and are not intended to limit the scope of the present invention.

1. A lipid-coated nanoparticle comprising:
(a) a nanoparticle;
(b) a plurality of payload molecules entrapped in the nanoparticle;
(c) a lipid coating around the nanoparticle and the plurality of payload molecules.

2. The lipid-coated nanoparticle of claim 1, wherein the lipid coating comprises an ionizable cationic lipid species, a cationic lipid species, an anionic lipid species, a neutral lipid species, a helper lipid species, or any combination thereof.

3. The lipid-coated nanoparticle of claim 1, wherein the lipid coating comprises a lipid mixture of 1,2-Dioleoyl-3-trimethylammonium propane (DOTAP) and 1,2-Dioleoyl-sn-glycero-3-phosphoethanolamine (DOPE).

4. The lipid-coated nanoparticle of claim 3, wherein the ratio of DOTAP:DOPE is 2.5:1 to 3.5:1, 2.7:1 to 3.3:1, 2.8:1 to 3.2:1, or 2.9:1 to 3.1:1.

5. The lipid-coated nanoparticle of claim 4, wherein the ratio of DOTAP:DOPE is 3:1.

6. The lipid-coated nanoparticle of claim 1, wherein the lipid coating comprises a lipid mixture of 1-Octylnonyl 8-[(hydroxyethyl)[6-oxo-6-(undecyloxy)hexyl]amino]octanoate (SM-102), 1,2-di-O-octadecenyl-3-trimethylammonium propane (DOTMA), and 1,2-Distearoyl-sn-glycero-3-phosphocholine (DSPC).

7. The lipid-coated nanoparticle of claim 6, wherein the ratio of SM-102:DOTMA:DSPC is 5:1:2 to 10:1:2, 5:2:4 to 10:2.5:6, 6:1:3 to 8:1:3, or 6:1:1 to 8:1:4.

8. The lipid-coated nanoparticle of claim 7, wherein the ratio of SM-102:DOTMA:DSPC is 7:1:2.

9. The lipid-coated nanoparticle of claim 1, wherein the lipid coating comprises a lipid mixture of BAE, DOTMA, and DSPC.

10. The lipid-coated nanoparticle of claim 9, wherein the ratio of BAE:DOTMA:DSPC is 5:1:2 to 15:1:2, 5:1:1 to 15:1:4, or 8:1:2 to 12:1:4.

11. The lipid-coated nanoparticle of claim 10, wherein the ratio of BAE:DOTMA:DSPC is 10.4:1:2.

12. The lipid-coated nanoparticle of claim 1, wherein the lipid coating comprises a lipid mixture of KT-001, DOTMA, and DSPC.

13. The lipid-coated nanoparticle of claim 12, wherein the ratio of KT-001:DOTMA:DSPC is 5:1:2 to 15:1:2, 5:1:1 to 15:1:4, or 8:1:2 to 12:1:4.

14. The lipid-coated nanoparticle of claim 13, wherein the ratio of KT-001:DOTMA:DSPC is 7.9:1:2.

15. The lipid-coated nanoparticle of any one of claims 1-14, wherein the nanoparticle is a lipid-based nanoparticle.

16. The lipid-coated nanoparticle of claim 15, wherein the nanoparticle is a lipid nanoparticle (LNP).

17. The lipid-coated nanoparticle of claim 15, wherein the nanoparticle is a solid lipid nanoparticle (SLN).

18. The lipid-coated nanoparticle of claim 15, wherein the nanoparticle is an emulsion nanoparticle.

19. The lipid-coated nanoparticle of any one of claims 15-18, wherein the nanoparticle comprises an ionizable cationic lipid, a helper lipid, and optionally a cholesterol and/or a PEG.

20. The lipid-coated nanoparticle of any one of claims 15-19, wherein the lipid nanoparticle comprises a neutral lipid and optionally a helper lipid.

21. The lipid-coated nanoparticle of any one of claims 15-19, wherein the lipid nanoparticle comprises a lipid mixture of KT-001, DSPC, cholesterol and DMG-PEG2000.

22. The lipid-coated nanoparticle of any one of claims 1-21, wherein the lipid coating is a liposome containing the nanoparticle.

23. The lipid-coated nanoparticle of claim 22, wherein a hydrophilic drug is in the aqueous core of the liposome in addition to the nanoparticle.

24. The lipid-coated nanoparticle of claim 22, wherein a hydrophobic drug is in the lipid bilayer of the liposome and the nanoparticle is in the aqueous core of the liposome.

25. The lipid-coated nanoparticle of any one of claims 1-21, wherein the lipid coating is a lipid micelle containing the nanoparticle.

26. The lipid-coated nanoparticle of claim 25, wherein a hydrophobic drug is in the hydrophobic core of the micelle in addition to the nanoparticle.

27. The lipid-coated nanoparticle of any one of claims 1-26, wherein the payload molecules comprise imaging agents, small molecules, or therapeutic agents, optionally wherein the therapeutic agents are small molecules or large molecules.

28. The lipid-coated nanoparticle of claim 27, wherein the payload molecules comprise therapeutic agents.

29. The lipid-coated nanoparticle of any one of claims 1-28, wherein the payload molecules comprise DNA or RNA.

30. A pharmaceutical composition comprising the lipid-coated nanoparticle of any one of claims 1-29, and a pharmaceutically acceptable excipient.

31. A method for manufacturing lipid-coated lipid nanoparticles (LNPs) comprising:
(a) dissolving at least one payload molecule into a first solution or a second solution, wherein the first solution comprises an aqueous phase and the second solution comprises an organic phase and a plurality of molecules capable of self-assembly, and wherein the first and second solutions are miscible;
(b) mixing the first solution and the second solution using microfluidics to obtain lipid nanoparticles encapsulating the at least one payload molecule under conditions suitable for LNP formation, thereby forming LNPs;
(c) purifying said LNPs;
(d) adjusting LNP concentration in an aqueous phase; and
(e) mixing said LNPs and a third solution using microfluidics to obtain lipid-coated LNPs, wherein the third solution comprises an organic phase and a plurality of molecules capable of self-assembly, and the third solution contains the same or different molecules as the second solution.

32. A method for manufacturing lipid-coated lipid nanoparticles (LNPs) comprising mixing LNPs with a third solution using microfluidics to obtain lipid-coated LNPs, wherein:
the LNPs were formed by mixing a first solution and a second solution using microfluidics under conditions suitable for LNP formation to obtain lipid nanoparticles encapsulating at least one payload molecule;
the first solution comprises an aqueous phase and the second solution comprises an organic phase and a plurality of molecules capable of self-assembly;
the first and second solutions are miscible;
the at least one payload molecule was dissolved in the first solution or the second solution;
the third solution comprises an organic phase and a plurality of molecules capable of self-assembly, and the third solution contains the same or different molecules as the second solution.

33. A method for manufacturing lipid-coated lipid nanoparticles (LNPs) comprising:
(a) preparing a first solution comprising an aqueous phase;
(b) preparing a second solution comprising an organic phase and a plurality of molecules capable of self-assembly, and wherein the first and second solutions are miscible;
(c) dissolving at least one payload molecule into the first or second solution;
(d) mixing said first and second solutions using microfluidics to obtain lipid nanoparticles encapsulating said payload under conditions suitable for LNP formation;
(e) purifying said LNPs;
(f) adjusting LNP concentration in aqueous phase;
(g) preparing a third solution comprising an organic phase and a plurality of molecules capable of self-assembly, wherein the third solution contains the same or different molecules as the second solution; and
(h) mixing said LNPs and third solution using microfluidics to obtain lipid-coated LNPs.

34. The method of any one of claims 31-33, further comprising purifying said lipid-coated LNPs.

35. The method of any one of claims 31-34, wherein the payload molecules comprise DNA or RNA.

36. The method of claim 35, wherein the payload molecules comprise mRNA.

37. The method of any one of claims 31-34, wherein the payload molecules comprise imaging agents.

38. The method of any one of claims 31-34, wherein the payload molecules comprise small molecules.

39. The method of any one of claims 31-38, wherein the aqueous phase is an aqueous buffer.

40. The method of any one of claims 31-39, wherein the organic phase of the second solution comprises a water-miscible organic solvent.

41. The method of claim 40, wherein the water-miscible organic solvent comprises ethanol.

42. The method of claim 40, wherein the water-miscible organic solvent comprises methanol.

43. The method of any one of claims 31-34, wherein the self-assembling molecules include at least a lipid component comprised of at least one species of lipid molecule.

44. The method of claim 43, wherein the at least one species of lipid molecule is selected from an ionizable cationic lipid species, a cationic lipid species, an anionic lipid species, a neutral lipid species, and a helper lipid species.

45. The method of any one of claim 31-34, 43, or 44, wherein said second solution includes two or three species of lipid molecules, wherein the species of lipid molecules are selected from an ionizable cationic lipid species, a cationic lipid species, an anionic lipid species, a neutral lipid species, a helper lipid species, or any combination thereof.

46. The method of any one of claim 31-34, 43, or 44, wherein said third solution includes two or three species of lipid molecules, wherein the species of lipid molecules are selected from an ionizable cationic lipid species, a cationic lipid species, an anionic lipid species, a neutral lipid species, a helper lipid species, or any combination thereof.

47. A method for transfecting a cell comprising contacting a cell with the lipid-coated nanoparticle of any one of claims 1-29 or the pharmaceutical composition of claim 30.

48. The method of claim 47, wherein the transfection is in-vitro.

49. The method of claim 47, wherein the transfection is in-vivo.

50. A method for administering a payload to a subject, comprising administering the lipid-coated nanoparticle of any one of claims 1-29 or the pharmaceutical composition of claim 30 to a subject in need thereof.
